(19) United States
(12) Patent Application Publication
(10) Pub. No.: US 2011/0038815 A1
(43) Pub. Date: Feb. 17, 2011

(54) DIMERIC BENZYLIDENE MALONATES

(75) Inventors: Markus Hansch, Lorrach (DE); Thomas Ehlis, Freiburg (DE); Barbara Wagner, Lorrach (DE)

Correspondence Address: BASF Corporation;Patent Department, 500 White Plains Road, P.O. Box 2005, Tarrytown, NY, 10591, US

(73) Assignee: BASF SE, Ludwigshafen (DE)

(21) Appl. No.: 12/867,777

(22) PCT Filed: Mar. 4, 2009
(86) PCT No.: PCT/EP2009/052531

§ 371 Date: Nov. 3, 2010

(30) Foreign Application Priority Data
Mar. 13, 2008 (EP) 08152707.9

(51) Int. Cl. A61K 8/37; C07C 67/31; A61Q 17/04

(52) U.S. Cl. 424/59; 560/55; 514/533

(57) ABSTRACT

Disclosed is the use of benzylidene malonates of formula (1), wherein R₁ and R₂, independently from each other are hydrogen; OH; C₁-C₁₂alkyl; C₁-C₁₂alkoxy; C₁-C₁₂alkylamino; C₁-C₁₂dialkylamino; water-solubilizing groups, selected from carboxylate, sulfonates or ammonium radicals; R₃ is COOR₅; COR₅; CONR₅R₆; CN; or SO₂R₇, R₄ is COOR₈; COR₉; CONR₈R₉; CN; or SO₂R₁₀; R₅, R₆, R₇, R₈, R₉ and R₁₀ independently from each other are C₁-C₁₈alkyl; unsubstituted or by one or more than one C₁-C₄alkyl substituted C₃-C₁₂-cycloalkyl; unsubstituted or C₁-C₆alkyl-substituted C₆-C₂₀aryl; organosilanyl, or organosiloxanyl; A is —C₁-C₂₀alkylene-, —CH₂CH(OH)CH₂—, —C₅-C₂₀cycloalkylene, —C₂-C₂₀alkenylene-, —C₅-C₁₀arylene-, or —C₅-C₁₀arylene-(C₁-C₁₀alkylene)- which are unsubstituted or substituted, straight-chain or branched, optionally interrupted by one or more than one heteroatoms selected from —O, —S— or —NH—; for the protection of human and animal hair and skin against UV radiation.

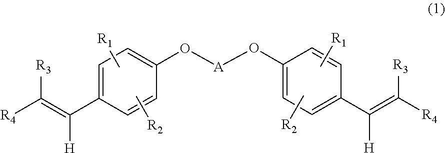

The present invention relates to the use of dimeric benzylidene malonates for cosmetic preparations.

It is well known that ultraviolet radiation (light) is harmful to human skin. Depending on the wavelength the UV radiation causes different types of skin damage. UV-B radiation (about 290 to about 320 nm) is responsible for sunburn and can cause skin cancer. UV-A radiation (about 320 to about 400 nm) while producing tanning of the skin, contributes also to sunburn and the induction of skin cancers. Moreover, the harmful effects of the UV-B radiation may be aggravated by UV-A radiation.

Therefore, an effective sunscreen formulation preferably comprises both at least one UV-A and UV-B filter and a broad band UV filter covering the full range of about 290 nm to about 400 nm to prevent the human skin from damaging by the sunlight.

Besides their screening power on solar radiation, UV filters must also have good resistance to water and perspiration, and also satisfactory photostability.

Unfortunately, many effective organic UV filters have a poor oil-solubility at a certain concentration and tend to crystallization. As a consequence the UV protection efficacy is significantly decreased.

Moreover the oil soluble UV filters should be included in cosmetic sun care products without any impact on the sensorial characteristic of the emulsion. For that reason the optimal distribution of the UV absorber within the hydro-lipid film left on the skin after spreading should be guaranteed.

It is therefore an object of the present invention to find UV absorber formulations which have improved properties regarding the UV absorber.

Surprisingly it has been found that specific dimeric benzylidene malonates have very good properties as cosmetic UV-B absorbers.

Therefore, the present invention relates to the use of benzylidene malonates of formula

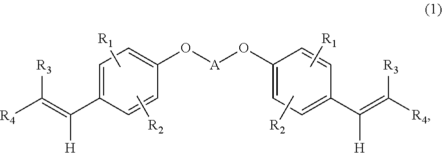

wherein
R₁ and R₂, independently from each other are hydrogen; OH; C₁-C₁₂alkyl; C₁-C₁₂alkoxy; C₁-C₁₂alkylamino; C₁-C₁₂dialkylamino; water-solubilizing groups selected from carboxylate, sulfonates and ammonium radicals;
R₃ is COOR₅, COR₅; CONR₅R₆; CN; or SO₂R₇;
R₄ is COOR₈; COR₉; CONR₈R₉; CN; or SO₂R₁₀;
R₅, R₆, R₇, R₈, R₉ and R₁₀ independently from each other are C₁-C₁₈alkyl; unsubstituted or by one or more than one C₁-C₄alkyl substituted C₃-C₁₂-cycloalkyl; unsubstituted or C₁-C₆alkyl-substituted C₆-C₂₀aryl; organosilanyl; or organosiloxanyl;
A is —C₁-C₂₀alkylene-, —CH₂CH(OH)CH₂—, —C₅-C₂₀cycloalkylene, —C₂-C₂₀alkenylene-, —C₅-C₁₀arylene-, or —C₅-C₁₀arylene-(C₁-C₁₀alkylene)- which are unsubstituted or substituted, straight-chain or branched, optionally interrupted by one or more than one heteroatoms selected from —O, —S— and —NH—;

for the protection of human and animal hair and skin against UV radiation.

C₁-C₁₈Alkyl denotes straight-chain and branched hydrocarbon radicals, for example methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetra-methylbutyl, 1-methylheptyl, 3-methylheptyl, n-octyl, 2-ethylhexyl, 1,1,3-trimethylhexyl, 1,1,3,3-tetramethylpentyl, nonyl, decyl, undecyl, 1-methylundecyl, dodecyl, tridecyl, tetradecyl, pentadecyl or hexadecyl or octadecyl.

Cycloalkyl is unsubstituted or by one or more than one C₁-C₄alkyl substituted C₃-C₁₂-cycloalkyl, for example cyclopropyl, cyclobutyl, cyclopentyl, cycloheptyl, cyclooctyl, cyclononyl, cyclodecyl, cycloundecyl, cyclodocecyl, 1-isopropyl-4-methyl-cyclohexyl (DL-menthyl) and most preferably cyclohexyl.

C₁-C₂₀alkylene may be straight-chain, branched, or, from C₅alkyl upwards, monocyclic or polycyclic, and may be interrupted by hetero atoms, such as such as —O—, —S—, —CO—, —NH—, NRₓ, —OCO—, —CO(ORₓ)—, —CONRₓ—, —(Rₓ)NC(O)—; wherein Rₓ is C₁-C₁₂alkyl.

C₁-C₂₀alkylene is for example methylene, ethylene, propylene, isopropylene, n-butylene, sec-butylene, tert-butylene, n-pentylene, 2-pentylene 3-pentylene, 2,2′-dimethylpropylene, cyclopentylene, cyclohexylene, n-hexylene, n-octylene, 1,1′,3,3′-tetramethylbutylene, 2-ethylhexylene, nonylene or decylene, dodecylene, undecylene or eicosylene;

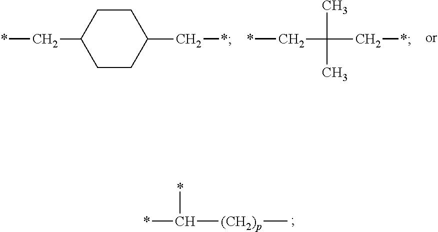

wherein p is a number from 0 to 12.

C₆-C₁₀arylene is for example phenyl or naphthyl.

Aryl-alkylene is for example C₅-C₁₀aryl-C₁-C₁₀alkylene, C₆-C₁₀aryl-C₁-C₂alkylene, alkyl-arylene is for example C₁-C₁₀alkyl-C₅-C₁₀arylene or C₁-C₂alkyl-C₆-C₁₀arylene.

C₅-C₁₀cycloalkylene is for example cyclopentylene, cyclohexylene, morpholylene or piperidinylene.

In formula (1)

R₅ R₆, R₇, R₈, R₉ and R₁₀ are preferably C₁-C₁₀alkyl.

R₁ and R₂ in formula (1) are preferably hydrogen; or a solubilizing group selected from carboxylate, sulfonate and ammonium residues.

Preferred compounds correspond to formula

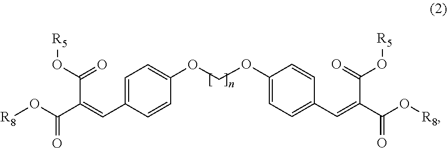

wherein

R₅ and R₈ are defined as in formula (1); and

n is a number from 1 to 20.

Most preferred are compounds of formula (2), wherein
R₅ and R₈ independently from each other are methyl; ethyl; propyl; isopropyl; n-butyl; sec-butyl; isobutyl; 2-methylbutyl; 2-ethylhexyl; SiMe₃; CH₂SiMe₃; CH₂CH₂SiMe₃; CH₂CH₂CH₂SiMe₃; or CH₂CH₂CH₂Si(OSiMe₃)₂Me; and
n is 1.

Most preferred are compounds of formula (2), wherein
R₅ and R₈ are methyl; ethyl; 2-methylbutyl; or 2-ethylhexy; and even more preferred are compounds of the present invention wherein in formula (2)
R₅ and R₈ have the same meaning.

The compounds of formula

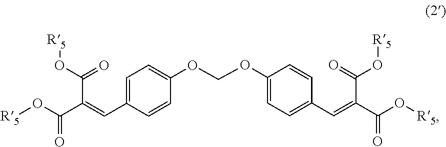

wherein
R′₅ is methyl, propyl, isopropyl, n-butyl, sec-butyl, isobutyl, 2-methylbutyl, 2-ethylhexyl, SiMe₃, CH₂SiMe₃, CH₂CH₂SiMe₃, CH₂CH₂CH₂SiMe₃ or CH₂CH₂CH₂Si(OSiMe₃)₂Me

are novel and represent a further subject of the present invention.

Examples of compounds of formula (2) are listed in the Table 1 below:

TABLE 1
Examples of dimeric benzylidene malonates according to the present invention

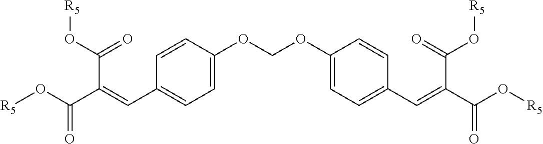

| No. | R₅ |
|---|---|
| DBM-01 | methyl |
| DBM-02 | ethyl |
| DBM-03 | n-propyl |
| DBM-04 | isopropyl |
| DBM-05 | n-butyl |
| DBM-06 | isobutyl |
| DBM-07 | tert-butyl |
| DBM-08 | n-hexyl |
| DBM-09 | n-octyl |
| DBM-10 | n-dodecyl |
| DBM-11 | 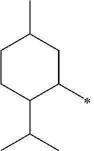 |
| DBM-12 | n-decyl |
| DBM-13 | 2-methyl-butyl |
| DBM-14 | 2,2-dimethylpropyl |
| DBM-15 | 3-methylbutyl |
| DBM-16 | 2-ethylhexyl |
| DBM-17 | Me₃Si—* |
| DBM-18 | Me₃Si—CH₂—* |
| DBM-19 | Me₃Si—(CH₂)₂—* |
| DBM-20 | Me₃Si—(CH₂)₃—* |
| DBM-21 | MeSi(OSiMe₃)₂CH₂CH₂CH₂—* |
| DBM-22 | 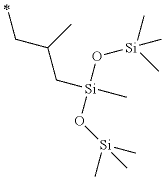 |
| DBM-23 | sec-butyl |
| DBM-24 | n-pentyl |

The benzylidene malonates according to the present invention are prepared in a manner known per se, as described, for example, in U.S. Pat. No. 3,706,700 or in Khimiko-Farmatsevticheskii Zhurnal 17(7), 801-6 (1983).

Preferred is a process for the preparation of the compounds of formula (2′), which comprises reacting the malonate (2a) with p-hydroxybenzaldehyde (2b) to the phenol intermediate (2c) in a Knoevenagel condensation ((a)) and linking two units of this intermediate with the dihaloalkane (2d) to the compound (2′) (step (b)) according to the following reaction scheme:

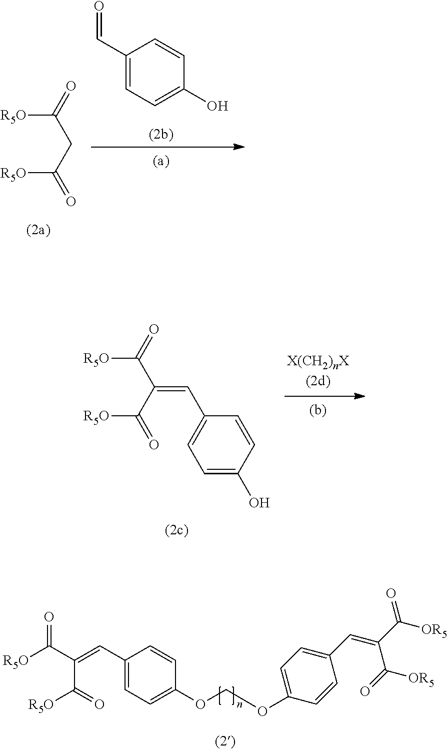

wherein

X is Cl, Br, or I; and

R₅ and n are defined as in formula (2).

Reaction step (a) is carried out under azeotropic removal of water in the presence of a catalyst (literature: Jones, Gurnos. Knoevenagel condensation. Organic Reactions (New York) 15, 204-599 (1967).

Solvents used in this step are for example benzene, toluene, o-xylene, m-xylene, p-xylene, chloroform, dichloromethane, ethanol, methanol, tetrahydrofurane, acetonitrile, ethyl acetate, CCl₄, cyclohexane, n-hexane, n-pentane, or ionic liquids as for example 1-methyl-3-butyl imidazolium bromide. Mixtures of solvents can also be used.

The reaction temperature is preferably between 0° C. and the reflux temperature of the solvent mixture, preferably between 0° C. and 180° C., and more preferably between 20° and 150° C.

The reaction time is preferably from 5 min to 72 h, and more preferably from 1 to 10 h.

The catalysts used in this reaction step are preferably primary, secondary or tertiary amines like piperidine, n-hexylamine, pyridine or triethylamine. The basic amines can be used as such or in combination with an acidic compound like acetic acid, benzoic acid or HCl. Suitable catalysts are any catalysts which are normally used in Knoevenagel reactions. Preferably are used catalysts such as a salt of an organic base with an organic acid, such as piperidine acetate.

Reaction step (b) is preferably carried out in the presence of a base, for example K₂CO₃, KOH, NaOH, Na₂CO₃, CsCO₃, K₃PO₄, Na₃PO₄, CaCO₃, LiOH, NaH, KH, LiH, amines like triethylamine, pyridine or diisopropylethylamine and a catalyst.

The alkylating agents X(CH₂)ₙX (X═Cl, I or Br) used in this step are for example 1,2-dibromoethane, 1,3-dibromopropane, 1,4-dibromobutane, 1,5-dibromopentane, dichloromethane, dibromomethane, bromochloromethane or diiodomethane.

Solvents used in this step are for example CH₂Cl₂, DMF, NMP, acetone, methylethylketone, acetonitrile, dioxane, THF, HMPA, diethyl ether, water, ionic liquids as for example 1-methyl-3-butyl imidazolium bromide or combinations of the mentioned solvents.

The reaction temperature is preferably between −10° C. and the reflux temperature of the solvent selected, more preferably between 0° C. and 180° C. and most preferably between 20 and 150° C.

The reaction time is preferably from 5 min to 150 h, and more preferably from 1 to 72 h.

The catalysts used in this step are preferably phase transfer catalysts like crown ether [18]crown-6, polyethylenglycol, salts like benzyl trimethyl ammonium chloride, tetrabutylammonium chloride, tetrabutylammonium bromide (TBAB), tributylmethylammonium chloride, alkyl benzyl dimethyl ammonium chloride, quaternary phosphonium salts like tetramethylphosphonium bromide or tetrabutylphosphonium bromide.

Reaction step (b) can be carried out without use of a catalyst if dibromomethane or bromochloromethane are used as alkylating agents. Reaction step (b) can be carried out without use of a catalyst if the reaction is carried out under a pressure of p>1 bar, for example if the reaction is carried out using an autoclave.

Further preferred is a process for the preparation of the compounds of formula (2′), which comprises reacting the malonate (2a) with the p-hydroxybenzaldehyde (2b) to the phenol intermediate (2c) in a Knoevenagel condensation (step (a)) and linking two units of this intermediate with a dihaloalkane (2d) to the compound (2′) (step (b)) and subsequent transesterification with the alcohol R₈OH to the compound (2′) (step (c)) according to following reaction scheme:

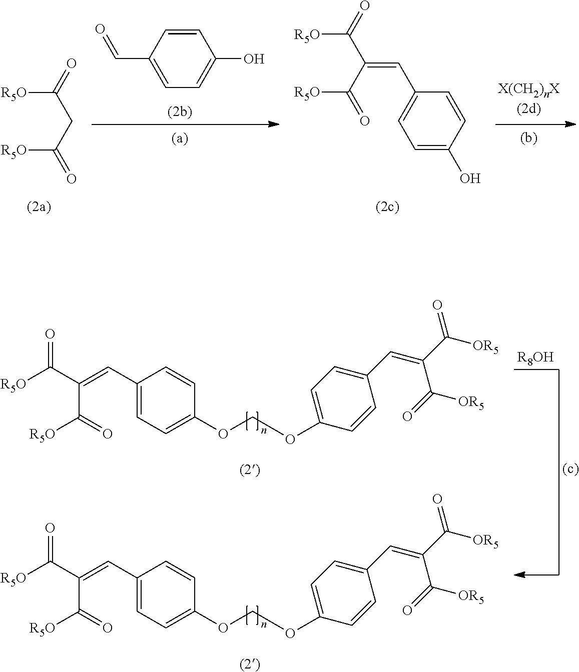

wherein

X is Cl, Br or I; and

R₅, R₈ and n are defined as in formula (2).

Steps (a) and (b) correspond to the reaction steps as described above.

Step (c) is a transesterification with a suitable alcohol in the presence of a catalyst.

Solvents used in step (c) are for example alcohol; benzene, toluene, o-xylene, m-xylene, p-xylene, chloroform, dichloromethane, ethanol, methanol, tetrahydrofurane, acetonitrile, ethyl acetate, CCl₄, cyclohexane, n-hexane, n-pentane, ionic liquids as for example 1-methyl-3-butyl imidazolium bromide or mixtures thereof.

The reaction temperature is preferably between 0° and 250° C. and more preferably between 30° and 180° C.

The reaction time is preferably from 5 min to 72 h and more preferably from 1 to 10 h.

Catalysts used in this step are preferably Broenstedt acids like HCl, H₂SO₄, p-toluene sulfonic acid, acetic acid, acidic ion exchange resins, Lewis acids like Ti(OiPr)₄, Ti(OEt)₄, Bu₂SnO, Broenstedt bases like NaOMe, NaOEt, LiOH, NaNH₂, lithium acetate or Al(OiPr)₃. Suitable are any catalysts which are normally used for transesterification reactions.

Preferred is also a process for the preparation of the compounds of formula (2′), which comprises linking two molecules of p-hydroxybenzaldehyde (2b) with the dihaloalkane (2d) to give the intermediate (2e) (step (a)) and subsequent Knoevenagel condensation of this intermediate with the malonate (2a) (step (b)) according to the following reaction scheme:

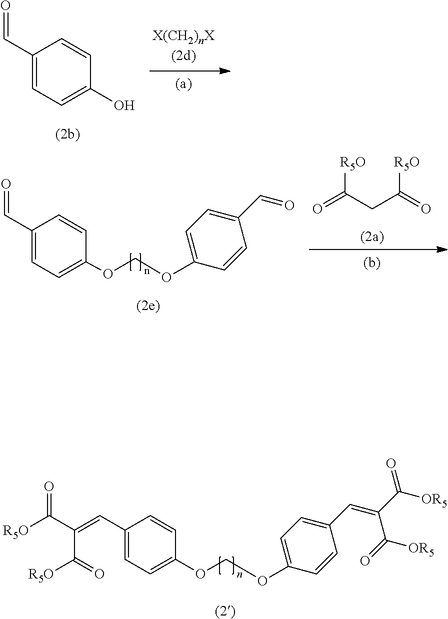

wherein

X is Cl, Br or I; and

R₅ and n are defined as in formula (2).

The reaction of p-hydroxybenzaldehyde with the dihaloalkane X(CH₂)ₙX (step (a)) can be carried out as described above and is described in H. B. Donahoe et al, Journal of Organic Chemistry 26, 474-6 (1961).

Step (b) corresponds to the Knoevenagel condensation as described above.

The dimeric benzylidene malonates according to formula (1) are suitable especially as UV filters, that is to say for the protection of organic materials that are sensitive to ultraviolet light, especially human and animal skin and hair, against the action of UV radiation. Such compounds are accordingly suitable as light-protective agents in cosmetic, pharmaceutical and veterinary medicine preparations. Such compounds are preferably used in the dissolved state.

The invention accordingly relates also to a cosmetic preparation comprising at least one compound of formula (1), and cosmetically tolerable carriers or adjuvants.

The cosmetic preparation may also comprise, in addition to the UV absorber according to the invention, one or more further UV protective agents of the following substance classes:
1. p-aminobenzoic acid derivatives, salicylic acid derivatives, benzophenone derivatives, dibenzoylmethane derivatives, diphenylacrylates, 3-imidazol-4-ylacrylic acid and esters; benzofuran derivatives, polymeric UV absorbers, cinnamic acid derivatives, camphor derivatives, hydroxyphenyltriazine compounds, benzotriazole compounds, trianilino-s-triazine derivatives, 2-phenylbenzimidazole-5-sulfonic acid and salts thereof; menthyl o-aminobenzoate; or TiO₂ (variously encapsulated), ZnO and mica.

The UV absorbers described in “Sunscreens”, Eds. N. J. Lowe, N. A. Shaath, Marcel Dekker, Inc., New York and Basle or in Cosmetics & Toiletries (107), 50ff (1992) also can be used as additional UV protective substances.

Special preference is given to the light-protective agents indicated in the following Table:

| INCI | Chemical Name | CAS No. |
|---|---|---|
| 3-BENZYLIDENE CAMPHOR | 1,7,7-trimethyl-3-(phenylmethylene)- | 15087-24-8 |
| | bicyclo[2.2.1]heptan-2-one | |
| 4-METHYLBENZYLIDENE | (+/−)-1,7,7-trimethyl-3-[(4-methylphenyl)- | 36861-47-9 |
| CAMPHOR | methylene]bicyclo[2.2.1]heptan-2-one | |
| BENZOPHENONE-10 | 2-hydroxy-4-methoxyphenyl)-(4-methyl- | 1641-17-4 |
| | phenyl)methanone | |
| BENZOPHENONE-1 | 2,4-dihydroxybenzophenone | 131-56-6 |
| BENZOPHENONE-2 | 2,2′,4,4′-tetrahydroxybenzophenone | 131-55-5 |
| BENZOPHENONE-3 | 2-hydroxy-4-methoxybenzophenone | 131-57-7 |
| BENZOPHENONE-4 | 2-hydroxy-4-methoxybenzophenone-5- | 4065-45-6 |
| | sulfonic acid | |
| BENZOPHENONE-6 | 2,2′-dihydroxy-4,4′-dimethoxybenzophenone | 131-54-4 |
| BENZOPHENONE-8 | 2,2′-dihydroxy-4-methoxybenzophenone | 131-53-3 |
| BENZYLIDENE CAMPHOR | alpha-(2-oxoborn-3-ylidene)-toluene-4- | 56039-58-8 |
| SULFONIC ACID | sulfonic acid and its salts | |
| BUTYL METHOXY- | 1-[4-(1,1-dimethylethyl)phenyl]-3-(4- | 70356-09-1 |
| DIBENZOYLMETHANE | methoxyphenyl)propane-1,3-dione | |
| CAMPHOR BENZALKONIUM | methyl N,N,N-trimethyl-4-[(4,7,7-trimethyl- | 52793-97- |
| METHOSULFATE | 3-oxobicyclo[2,2,1]hept-2-ylidene)methyl]- | |
| | anilinium sulfate | |
| CINOXATE | 2-ethoxyethyl p-methoxycinnamate | 104-28-9 |
| DEA-METHOXYCINNAMATE | diethanolamine salt of p-methoxy- | 56265-46-4 |
| | hydrocinnamate | |
| DIISOPROPYL METHYL | 2-propenoic acid, 3-[2,4-bis(1- | 32580-71-5 |
| CINNAMATE | methylethyl)phenyl]-, methyl ester | |
| DIPROPYLENE GLYCOL | dipropylene glycol salicylate | 7491-14-7 |
| SALICYLATE | | |
| ETHYL | ethyl 4-bis(2-hydroxypropyl)-amino- | 58882-17-0 |
| DIHYDROXYPROPYL PABA | benzoate | |
| ETHYL | ethyl 3- [2,4-bis(1-methylethyl)phenyl]acrylate | 32580-72-6 |
| DIISOPROPYLCINNAMATE | | |
| ETHYL | ethyl p-methoxycinnamate | 1929-30-2 |
| METHOXYCINNAMATE | | |
| GLYCERYL OCTANOATE | | |
| DIMETHOXYCINNAMATE | | |
| GLYCERYL PABA | glyceryl 1-(4-aminobenzoate) | 136-44-7 |
| HOMOSALATE | 3,3,5-trimethylcyclohexyl-2-hydroxy- | 118-56-9 |
| | benzoate | |
| ISOAMYL p-METHOXY- | isopentyl p-methoxycinnamate | 71617-10-2 |
| CINNAMATE | | |
| ISOPROPYL | 1-[4-(1-methylethyl)phenyl]-3-phenyl- | 63250-25-9 |
| DIBENZOYLMETHANE | propane-1,3-dione | |
| ISOPROPYL | isopropyl p-methoxycinnamate | 5466-76-2 |
| METHOXYCINNAMATE | | |
| LAWSONE | 2-hydroxy-1,4-naphthoquinone | 83-72-7 |
| MENTHYL ANTHRANILATE | menthyl o-aminobenzoate | 134-09-8 |
| MENTHYL SALICYLATE | menthyl salicylate | 89-46-3 |
| OCTOCRYLENE | 2-ethylhexyl 2-cyano-3,3-diphenyl acrylate | 6197-30-4 |
| ETHYLHEXYL DIMETHYL | 2-ethylhexyl 4-(dimethylamino)benzoate | 21245-02-3 |
| PABA | | |
| ETHYLHEXYL | 2-ethylhexyl 4-methoxycinnamate | 5466-77-3 |
| METHOXYCINNAMATE | | |
| ETHYLHEXYL SALICYLATE | 2-ethylhexyl salicylate | 118-60-5 |
| ETHYLHEXYL TRIAZONE | benzoic acid, 4,4′,4″-(1,3,5-triazine-2,4,6- | 88122-99-0 |
| | triyltriimino)tris-, tris(2-ethylhexyl) ester; 2,4,6- | |
| | trianilino-(p-carbo-2′-ethylhexyl- | |
| | 1′-oxy)-1,3,5-triazine | |
| PABA | 4-aminobenzoic acid | 150-13-0 |
| PEG-25 PABA | benzoic acid, 4-amino-, ethyl ester, | 113010-52-9 |
| | polymer with oxirane | |
| PENTYL DIMETHYL PABA | amyl dimethyl PABA | 14779-78-3 |
| PHENYLBENZIMIDAZOLE | 2-phenyl-1H-benzimidazole-5-sulfonic acid | 27503-81-7 |
| SULFONIC ACID | | |
| POLYACRYLAMIDOMETHYL | | 113783-61-2 |
| BENZYLIDENE CAMPHOR | | |
| TEA-SALICYLATE | triethanolamine salicylate | 2174-16-5 |
| TEREPHTHALYLIDENE | 3,3′-(1,4-phenylenedimethylene)bis[7,7- | 90457-82-2 |
| DICAMPHOR SULFONIC | dimethyl-2-oxo-bicyclo[2.2.1]heptane-1- | |
| ACID | methanesulfonic acid] | |
| TITANIUM DIOXIDE | titanium dioxide | 13463-67-7 |
| DIGALLOYL TRIOLEATE | digalloyl trioleate | 17048-39-4 |
| ZINC OXIDE | zinc oxide | 1314-13-2 |
| Methylene bis-benzotriazolyl | 2,2′-methylene-bis[6-(2H-benzotriazol-2-yl)-4- | 103597-45-1 |
| tetramethylbutylphenol | (1,1,3,3-tetramethylbutyl)-phenol] | |
| Bis-ethylhexyloxyphenol | 2,4-bis{[4-(2-ethylhexyloxy)-2-hydroxy]- | 187393-00-6 |
| methoxyphenyltriazine | phenyl}-6-(4-methoxyphenyl)-(1,3,5)-triazine | |
| BISIMIDAZYLATE | 1H-benzimidazole-4,6-disulfonic acid, | 180898-37-7 |
| | 2,2′-(1,4-phenylene)bis-, disodium salt | |
| DIETHYLHEXYL BUTAMIDO | benzoic acid, 4,4′-[[6-[[4-[[(1,1-dimethylethyl)- | 154702-15-5 |
| TRIAZONE | amino]carbonyl]phenyl]amino]-1,3,5-triazine- | |
| | 2,4-diyl]diimino]bis-, bis(2-ethyl-hexyl) ester | |
| DROMETRIZOLE | phenol, 2-(2H-benzotriazol-2-yl)-4-methyl- | 155633-54-8 |
| TRISILOXANE | 6-[2-methyl-3-[1,3,3,3-tetramethyl-1-[(tri- | |
| | methylsilyl)oxy]disiloxanyl]propyl]- | |
| BENZYLIDENE MALONATE | alpha-(trimethylsilyl)-omega-(trimethyl-silyl- | 207574-74-1 |
| POLYSILOXANE | oxy)poly[oxy(dimethyl)silylene]-co-[oxy(methyl)(2- | |
| | {p-[2,2-bis(ethoxycarbonyl)vinyl]- | |
| | phenoxy}-1-methyleneethyl)silylene]-co- | |
| | [oxy(methyl)(2-{p-[2,2-bis(ethoxycarbonyl)- | |
| | vinyl]phenoxy}prop-1-enyl)silylene] | |
| | Diethylamino hydroxybenzoyl Hexyl Benzoate | 302776-68-7 |

Each of the above-mentioned light-protective agents, especially the light-protective agents in the above Table indicated as being preferred, can be used in admixture with the UV absorbers according to the invention. It will be understood in that connection that, in addition to the UV absorbers according to the invention, it is also possible for more than one of the additional light-protective agents to be used, for example, two, three, four, five or six further light-protective agents. Preference is given to the use of mixing ratios of UV absorbers according to the invention/further light-protective agents of from 1:99 to 99:1, especially from 1:95 to 95:1 and preferably from 10:90 to 90:10, based on weight. Of special interest are mixing ratios of from 20:80 to 80:20, especially from 40:60 to 60:40 and preferably of approximately 50:50. Such mixtures can be used, inter alia, to improve solubility or to increase UV absorption.

Appropriate mixtures can be used especially advantageously in a cosmetic composition according to the invention.

Suitable new UV filters are listed in Table 1 (compounds DBM-01-DBM-24).

Other typical ingredients in such formulations are preservatives, bactericides and bacteriostatic agents, perfumes, dyes, pigments, thickening agents, moisturizing agents, humectants, fats, oils, waxes or other typical ingredients of cosmetic and personal care formulations such as alcohols, poly-alcohols, polymers, electrolytes, organic solvents, silicon derivatives, emollients, emulsifiers or emulsifying surfactants, surfactants, dispersing agents, antioxidants, anti-irritants and anti-inflammatory agents etc.

The described cosmetic or pharmaceutical preparations are distinguished by excellent protection of human skin against the damaging effect of sunlight.

The following mixtures of soluble UV filters (Table 2) can be mixed together with the benzylidene malonates according to the present invention:

TABLE 2
List of oil soluble organic UV filter combinations

| | Abbreviation | | | | | | | | | |
|---|---|---|---|---|---|---|---|---|---|---|
| Combination Nr. | BP3 | BP4 | 3BC | BEMT | BMBM | DBT | DTS | EHT | MBC | PAMBC |
| UV SOL 1 | x | | | | | | | | | |
| UV SOL 2 | | x | | | | | | | | |
| UV SOL 3 | | | x | | | | | | | |
| UV SOL 4 | | | | x | | | | | | |
| UV SOL 5 | | | | | x | | | | | |
| UV SOL 6 | | | | | | x | | | | |
| UV SOL 7 | | | | | | | x | | | |
| UV SOL 8 | | | | | | | | x | | |
| UV SOL 9 | | | | | | | | | x | |
| UV SOL 10 | | | | | | | | | | x |
| UV SOL 11 | x | x | | | | | | | | |
| UV SOL 12 | x | | x | | | | | | | |
| UV SOL 13 | x | | | x | | | | | | |
| UV SOL 14 | x | | | | x | | | | | |
| UV SOL 15 | x | | | | | x | | | | |
| UV SOL 16 | x | | | | | | x | | | |
| UV SOL 17 | x | | | | | | | x | | |
| UV SOL 18 | x | | | | | | | | x | |
| UV SOL 19 | x | | | | | | | | | x |
| UV SOL 20 | | x | x | | | | | | | |
| UV SOL 21 | | x | | x | | | | | | |
| UV SOL 22 | | x | | | x | | | | | |
| UV SOL 23 | | x | | | | x | | | | |
| UV SOL 24 | | x | | | | | x | | | |
| UV SOL 25 | | x | | | | | | x | | |
| UV SOL 26 | | x | | | | | | | x | |
| UV SOL 27 | | x | | | | | | | | x |
| UV SOL 28 | | | x | x | | | | | | |
| UV SOL 29 | | | x | | x | | | | | |
| UV SOL 30 | | | x | | | x | | | | |
| UV SOL 31 | | | x | | | | x | | | |
| UV SOL 32 | | | x | | | | | x | | |
| UV SOL 33 | | | x | | | | | | x | |
| UV SOL 34 | | | x | | | | | | | x |
| UV SOL 35 | | | | x | x | | | | | |
| UV SOL 36 | | | | x | | x | | | | |
| UV SOL 37 | | | | x | | | x | | | |
| UV SOL 38 | | | | x | | | | x | | |
| UV SOL 39 | | | | x | | | | | x | |
| UV SOL 40 | | | | x | | | | | | x |
| UV SOL 41 | | | | | x | x | | | | |
| UV SOL 42 | | | | | x | | x | | | |
| UV SOL 43 | | | | | x | | | x | | |
| UV SOL 44 | | | | | x | | | | x | |
| UV SOL 45 | | | | | x | | | | | x |
| UV SOL 46 | | | | | | x | x | | | |
| UV SOL 47 | | | | | | x | | x | | |
| UV SOL 48 | | | | | | x | | | x | |
| UV SOL 49 | | | | | | x | | | | x |
| UV SOL 50 | | | | | | | x | x | | |
| UV SOL 51 | | | | | | | x | | x | |
| UV SOL 52 | | | | | | | x | | | x |
| UV SOL 53 | | | | | | | | x | x | |
| UV SOL 54 | | | | | | | | x | | x |
| UV SOL 55 | | | | | | | | | x | x |
| UV SOL 56 | x | x | x | | | | | | | |
| UV SOL 57 | x | x | | x | | | | | | |
| UV SOL 58 | x | x | | | x | | | | | |
| UV SOL 59 | x | x | | | | x | | | | |
| UV SOL 60 | x | x | | | | | x | | | |
| UV SOL 61 | x | x | | | | | | x | | |
| UV SOL 62 | x | x | | | | | | | x | |
| UV SOL 63 | x | x | | | | | | | | x |
| UV SOL 64 | x | | x | x | | | | | | |
| UV SOL 65 | x | | x | | x | | | | | |
| UV SOL 66 | x | | x | | | x | | | | |
| UV SOL 67 | x | | x | | | | x | | | |
| UV SOL 68 | x | | x | | | | | x | | |
| UV SOL 69 | x | | x | | | | | | x | |
| UV SOL 70 | x | | x | | | | | | | x |
| UV SOL 71 | x | | | x | x | | | | | |
| UV SOL 72 | x | | | x | | x | | | | |
| UV SOL 73 | x | | | x | | | x | | | |
| UV SOL 74 | x | | | x | | | | x | | |
| UV SOL 75 | x | | | x | | | | | x | |
| UV SOL 76 | x | | | x | | | | | | x |
| UV SOL 77 | x | | | | x | x | | | | |
| UV SOL 78 | x | | | | x | | x | | | |
| UV SOL 79 | x | | | | x | | | x | | |
| UV SOL 80 | x | | | | x | | | | x | |
| UV SOL 81 | x | | | | x | | | | | x |
| UV SOL 82 | x | | | | | x | x | | | |
| UV SOL 83 | x | | | | | x | | x | | |
| UV SOL 84 | x | | | | | x | | | x | |
| UV SOL 85 | x | | | | | | x | | | x |
| UV SOL 86 | x | | | | | | x | x | | |
| UV SOL 87 | x | | | | | | x | | x | |
| UV SOL 88 | x | | | | | | x | | | x |
| UV SOL 89 | x | | | | | | | x | x | |
| UV SOL 90 | x | | | | | | | x | | x |
| UV SOL 91 | x | | | | | | | | x | x |
| UV SOL 92 | | x | x | x | | | | | | |
| UV SOL 93 | | x | x | | x | | | | | |
| UV SOL 94 | | x | x | | | x | | | | |
| UV SOL 95 | | x | x | | | | x | | | |
| UV SOL 96 | | x | x | | | | | x | | |
| UV SOL 97 | | x | x | | | | | | x | |
| UV SOL 98 | | x | x | | | | | | | x |
| UV SOL 99 | | x | | x | x | | | | | |
| UV SOL 100 | | x | | x | | x | | | | |
| UV SOL 101 | | x | | x | | | x | | | |
| UV SOL 102 | | x | | x | | | | x | | |
| UV SOL 103 | | x | | x | | | | | x | |
| UV SOL 104 | | x | | x | | | | | | x |
| UV SOL 105 | | x | | | x | x | | | | |
| UV SOL 106 | | x | | | x | | x | | | |
| UV SOL 107 | | x | | | x | | | x | | |
| UV SOL 108 | | x | | | x | | | | x | |
| UV SOL 109 | | x | | | x | | | | | x |
| UV SOL 110 | | x | | | | x | x | | | |
| UV SOL 111 | | x | | | | x | | x | | |
| UV SOL 112 | | x | | | | x | | | x | |
| UV SOL 113 | | x | | | | x | | | | x |
| UV SOL 114 | | x | | | | | x | x | | |
| UV SOL 115 | | x | | | | | x | | x | |
| UV SOL 116 | | x | | | | | x | | | x |
| UV SOL 117 | | x | | | | | | x | x | |
| UV SOL 118 | | x | | | | | | x | | x |
| UV SOL 119 | | x | | | | | | | x | x |
| UV SOL 120 | | | x | x | x | | | | | |
| UV SOL 121 | | | x | x | | x | | | | |
| UV SOL 122 | | | x | x | | | x | | | |
| UV SOL 123 | | | x | x | | | | x | | |
| UV SOL 124 | | | x | x | | | | | x | |
| UV SOL 125 | | | x | x | | | | | | x |
| UV SOL 126 | | | x | | x | x | | | | |
| UV SOL 127 | | | x | | x | | x | | | |
| UV SOL 128 | | | x | | x | | | x | | |
| UV SOL 129 | | | x | | x | | | | x | |
| UV SOL 130 | | | x | | x | | | | | x |
| UV SOL 131 | | | x | | | x | x | | | |
| UV SOL 132 | | | x | | | x | | x | | |
| UV SOL 133 | | | x | | | x | | | x | |
| UV SOL 134 | | | x | | | x | | | | x |
| UV SOL 135 | | | x | | | | x | x | | |
| UV SOL 136 | | | x | | | | x | | x | |
| UV SOL 137 | | | x | | | | x | | | x |
| UV SOL 138 | | | x | | | | | x | x | |
| UV SOL 139 | | | x | | | | | x | | x |
| UV SOL 140 | | | x | | | | | | x | x |
| UV SOL 141 | | | | x | x | x | | | | |
| UV SOL 142 | | | | x | x | | x | | | |
| UV SOL 143 | | | | x | x | | | x | | |
| UV SOL 144 | | | | x | x | | | | x | |
| UV SOL 145 | | | | x | x | | | | | x |
| UV SOL 146 | | | | x | | x | x | | | |
| UV SOL 147 | | | | x | | x | | x | | |
| UV SOL 148 | | | | x | | x | | | x | |
| UV SOL 149 | | | | x | | x | | | | x |
| UV SOL 150 | | | | x | | | x | x | | |
| UV SOL 151 | | | | x | | | x | | x | |
| UV SOL 152 | | | | x | | | x | | | x |
| UV SOL 153 | | | | x | | | | x | x | |
| UV SOL 154 | | | | x | | | | x | | x |
| UV SOL 155 | | | | x | | | | | x | x |
| UV SOL 156 | | | | | x | x | x | | | |
| UV SOL 157 | | | | | x | x | | x | | |
| UV SOL 158 | | | | | x | x | | | x | |
| UV SOL 159 | | | | | x | x | | | | x |
| UV SOL 160 | | | | | x | | x | x | | |
| UV SOL 161 | | | | | x | | x | | x | |
| UV SOL 162 | | | | | x | | x | | | x |
| UV SOL 163 | | | | | x | | | x | x | |
| UV SOL 164 | | | | | x | | | x | | x |
| UV SOL 165 | | | | | x | | | | x | x |
| UV SOL 166 | | | | | | x | x | x | | |
| UV SOL 167 | | | | | | x | x | | x | |
| UV SOL 168 | | | | | | x | x | | | x |
| UV SOL 169 | | | | | | x | | x | x | |
| UV SOL 170 | | | | | | x | | x | | x |
| UV SOL 171 | | | | | | x | | | x | x |
| UV SOL 172 | | | | | | | x | x | x | |
| UV SOL 173 | | | | | | | x | x | | x |
| UV SOL 174 | | | | | | | x | | x | x |
| UV SOL 175 | | | | | | | | x | x | x |
| UV SOL 176 | x | x | x | x | | | | | | |
| UV SOL 177 | x | x | x | | x | | | | | |
| UV SOL 178 | x | x | x | | | x | | | | |
| UV SOL 179 | x | x | x | | | | x | | | |
| UV SOL 180 | x | x | x | | | | | x | | |
| UV SOL 181 | x | x | x | | | | | | x | |
| UV SOL 182 | x | | x | x | x | | | | | x |
| UV SOL 183 | x | | x | x | | x | | | | |
| UV SOL 184 | x | | x | x | | | x | | | |
| UV SOL 185 | x | | x | x | | | | x | | |
| UV SOL 186 | x | | x | x | | | | | x | |
| UV SOL 187 | x | | x | x | | | | | | x |
| UV SOL 188 | x | | | x | x | x | | | | |
| UV SOL 189 | x | | | x | x | | x | | | |
| UV SOL 190 | x | | | x | x | | | x | | |
| UV SOL 191 | x | | | x | x | | | | x | |
| UV SOL 192 | x | | | x | x | | | | | x |
| UV SOL 193 | x | | | | x | x | x | | | |
| UV SOL 194 | x | | | | x | x | | x | | |
| UV SOL 195 | x | | | | x | x | | | x | |
| UV SOL 196 | x | | | | x | x | | | | x |
| UV SOL 197 | x | | | | | x | x | x | | |
| UV SOL 198 | x | | | | | x | x | | x | |
| UV SOL 199 | x | | | | | x | x | | | x |
| UV SOL 200 | x | | | | | | x | x | x | |
| UV SOL 201 | x | | | | | | x | x | | x |
| UV SOL 202 | x | | | | | | | x | x | x |
| UV SOL 203 | | x | x | x | x | | | | | |
| UV SOL 204 | | x | x | x | | x | | | | |
| UV SOL 205 | | x | x | x | | | x | | | |
| UV SOL 206 | | x | x | x | | | | x | | |
| UV SOL 207 | | x | x | x | | | | | x | |
| UV SOL 208 | | x | x | x | | | | | | x |
| UV SOL 209 | | x | | x | x | | x | | | |
| UV SOL 210 | | x | | x | x | | | x | | |
| UV SOL 211 | | x | | x | x | | | | x | |
| UV SOL 212 | | x | | x | x | | | | | x |
| UV SOL 213 | | x | | | x | x | x | | | |
| UV SOL 214 | | x | | | x | x | | x | | |
| UV SOL 215 | | x | | | x | x | | | x | |
| UV SOL 216 | | x | | | x | x | | | | x |
| UV SOL 217 | | x | | | | x | x | x | | |
| UV SOL 218 | | x | | | | x | x | | x | |
| UV SOL 219 | | x | | | | x | x | | | x |
| UV SOL 220 | | x | | | | | x | x | x | |
| UV SOL 221 | | x | | | | | x | x | | x |
| UV SOL 222 | | x | | | | | | x | x | x |
| UV SOL 223 | | | x | x | x | x | | | | |
| UV SOL 224 | | | x | x | x | | x | | | |
| UV SOL 225 | | | x | x | x | | | x | | |
| UV SOL 226 | | | x | x | x | | | | x | |
| UV SOL 227 | | | x | x | x | | | | | x |
| UV SOL 228 | | | x | | x | x | | x | | |
| UV SOL 229 | | | x | | x | x | | | x | |
| UV SOL 230 | | | x | | x | x | | | | x |
| UV SOL 231 | | | x | | | x | x | x | | |
| UV SOL 232 | | | x | | | x | x | | x | |
| UV SOL 233 | | | x | | | x | x | | | x |
| UV SOL 234 | | | x | | | | x | x | x | |
| UV SOL 235 | | | x | | | | x | x | | x |
| UV SOL 236 | | | x | | | | | x | x | x |
| UV SOL 237 | | | | x | x | x | x | | | |
| UV SOL 238 | | | | x | x | x | | x | | |
| UV SOL 239 | | | | x | x | x | | | x | |
| UV SOL 240 | | | | x | x | x | | | | x |
| UV SOL 241 | | | | x | | x | x | | x | |
| UV SOL 242 | | | | x | | x | x | | | x |
| UV SOL 243 | | | | x | | | x | x | x | |
| UV SOL 244 | | | | x | | | x | x | | x |
| UV SOL 245 | | | | x | | | | x | x | x |
| UV SOL 246 | | | | | x | x | x | x | | |
| UV SOL 247 | | | | | x | x | x | | x | |
| UV SOL 248 | | | | | x | x | x | | | x |
| UV SOL 249 | | | | | x | | x | x | | x |
| UV SOL 250 | | | | | x | | | x | x | x |
| UV SOL 251 | | | | | | x | x | x | x | |
| UV SOL 252 | | | | | | x | x | x | | x |
| UV SOL 253 | | | | | | | x | x | x | x |

Table 2 Abbreviations

| | | |
|---|---|---|
| BP3 | Benzophenone 3 | 131-57-7 |
| BP4 | Benzophenone-4 | 4065-45-6 |
| 3BC | 3-Benzydilene Camphor | 15087-24-8 |
| BEMT | Bis-Ethylhexyloxyphenol Methoxyphenyl Triazine | 103597-45-1 |
| BMBM | Butyl Methoxydibenzoylmethane | 70356-09-1 |
| DBT | Diethylhexyl Butamido Triazone | 154702-15-5 |
| DTS | Drometrizole Trisiloxane | 155633-54-8 |
| EHT | Ethylhexyl Triazone | 88122-99-0 |
| MBC | 4-Methylbenzylidene Camphor | 36861-47-9 |
| PAMBC | Polyacrylamido Methylbenzylidene Camphor | 147897-12-9 |

The following mixtures of oil miscible organic UV filters (Table 3) can be mixed together with the benzylidene malonates according to the present invention:

TABLE 3
List of oil miscible organic UV filter combinations

| | Abbreviation | | | | | | | |
|---|---|---|---|---|---|---|---|---|
| Combination Nr. | DHHB | EHDP | EHMC | EHS | HMS | IMC | OCR | PS15 |
| UV LIQ 1 | x | | | | | | | |
| UV LIQ 2 | | x | | | | | | |
| UV LIQ 3 | | | x | | | | | |
| UV LIQ 4 | | | | x | | | | |
| UV LIQ 5 | | | | | x | | | |
| UV LIQ 6 | | | | | | x | | |
| UV LIQ 7 | | | | | | | x | |
| UV LIQ 8 | | | | | | | | x |
| UV LIQ 9 | x | x | | | | | | |
| UV LIQ 10 | x | | x | | | | | |
| UV LIQ 11 | x | | | x | | | | |
| UV LIQ 12 | x | | | | x | | | |
| UV LIQ 13 | x | | | | | x | | |
| UV LIQ 14 | x | | | | | | x | |
| UV LIQ 15 | x | | | | | | | x |
| UV LIQ 16 | | x | x | | | | | |
| UV LIQ 17 | | x | | x | | | | |
| UV LIQ 18 | | x | | | x | | | |
| UV LIQ 19 | | x | | | | x | | |
| UV LIQ 20 | | x | | | | | x | |
| UV LIQ 21 | | x | | | | | | x |
| UV LIQ 22 | | | x | x | | | | |
| UV LIQ 23 | | | x | | x | | | |
| UV LIQ 24 | | | x | | | x | | |
| UV LIQ 25 | | | x | | | | x | |
| UV LIQ 26 | | | x | | | | | x |
| UV LIQ 27 | | | | x | x | | | |
| UV LIQ 28 | | | | x | | x | | |
| UV LIQ 29 | | | | x | | | x | |
| UV LIQ 30 | | | | x | | | | x |
| UV LIQ 31 | | | | | x | x | | |
| UV LIQ 32 | | | | | x | | x | |
| UV LIQ 33 | | | | | x | | | x |
| UV LIQ 34 | | | | | | x | x | |
| UV LIQ 35 | | | | | | x | | x |
| UV LIQ 36 | | | | | | | x | x |
| UV LIQ 37 | x | x | x | | | | | |
| UV LIQ 38 | x | x | | x | | | | |
| UV LIQ 39 | x | x | | | x | | | |
| UV LIQ 40 | x | x | | | | x | | |
| UV LIQ 41 | x | x | | | | | x | |
| UV LIQ 42 | x | x | | | | | | x |
| UV LIQ 43 | x | | x | x | | | | |
| UV LIQ 44 | x | | x | | x | | | |
| UV LIQ 45 | x | | x | | | x | | |
| UV LIQ 46 | x | | x | | | | x | |
| UV LIQ 47 | x | | x | | | | | x |
| UV LIQ 48 | x | | | x | x | | | |
| UV LIQ 49 | x | | | x | | x | | |
| UV LIQ 50 | x | | | x | | | x | |
| UV LIQ 51 | x | | | x | | | | x |
| UV LIQ 52 | x | | | | x | x | | |
| UV LIQ 53 | x | | | | x | | x | |
| UV LIQ 54 | x | | | | x | | | x |
| UV LIQ 55 | x | | | | | x | x | |
| UV LIQ 56 | x | | | | | x | | x |
| UV LIQ 57 | x | | | | | | x | x |
| UV LIQ 58 | | x | x | x | | | | |
| UV LIQ 59 | | x | x | | x | | | |
| UV LIQ 60 | | x | x | | | x | | |
| UV LIQ 61 | | x | x | | | | x | |
| UV LIQ 62 | | x | x | | | | | x |
| UV LIQ 63 | | x | | x | x | | | |
| UV LIQ 64 | | x | | x | | x | | |
| UV LIQ 65 | | x | | x | | | x | |
| UV LIQ 66 | | x | | x | | | | x |
| UV LIQ 67 | | x | | | x | x | | |
| UV LIQ 68 | | x | | | x | | x | |
| UV LIQ 69 | | x | | | x | | | x |
| UV LIQ 70 | | x | | | | x | x | |
| UV LIQ 71 | | x | | | | x | | x |
| UV LIQ 72 | | x | | | | | x | x |
| UV LIQ 73 | | | x | x | x | | | |
| UV LIQ 74 | | | x | x | | x | | |
| UV LIQ 75 | | | x | x | | | x | |
| UV LIQ 76 | | | x | x | | | | x |
| UV LIQ 77 | | | x | | x | x | | |
| UV LIQ 78 | | | x | | x | | x | |
| UV LIQ 79 | | | x | | x | | | x |
| UV LIQ 80 | | | x | | | x | x | |
| UV LIQ 81 | | | x | | | x | | x |
| UV LIQ 82 | | | x | | | | x | x |
| UV LIQ 83 | | | | x | x | x | | |
| UV LIQ 84 | | | | x | x | | x | |
| UV LIQ 85 | | | | x | x | | | x |
| UV LIQ 86 | | | | x | | x | x | |
| UV LIQ 87 | | | | x | | x | | x |
| UV LIQ 88 | | | | x | | | x | x |
| UV LIQ 89 | | | | | x | x | x | |
| UV LIQ 90 | | | | | x | x | | x |
| UV LIQ 91 | | | | | x | | x | x |
| UV LIQ 92 | | | | | | x | x | x |
| UV LIQ 93 | x | x | x | x | | | | |
| UV LIQ 94 | x | x | x | | x | | | |
| UV LIQ 95 | x | x | x | | | x | | |
| UV LIQ 96 | x | x | x | | | | x | |
| UV LIQ 97 | x | x | x | | | | | x |
| UV LIQ 98 | x | | x | x | | x | | |
| UV LIQ 99 | x | | x | x | | | x | |
| UV LIQ 100 | x | | x | x | | | | x |
| UV LIQ 101 | x | | | x | x | x | | |
| UV LIQ 102 | x | | | x | x | | x | |
| UV LIQ 103 | x | | | x | x | | | x |
| UV LIQ 104 | x | | | | x | x | x | |
| UV LIQ 105 | x | | | | x | x | | x |
| UV LIQ 106 | x | | | | | x | x | x |
| UV LIQ 107 | | x | x | x | x | | | |
| UV LIQ 108 | | x | x | x | | x | | |
| UV LIQ 109 | | x | x | x | | | x | |
| UV LIQ 110 | | x | x | x | | | | x |
| UV LIQ 111 | | x | | x | x | | x | |
| UV LIQ 112 | | x | | x | x | | | x |
| UV LIQ 113 | | x | | | x | x | x | |
| UV LIQ 114 | | x | | | x | x | | x |
| UV LIQ 115 | | x | | | | x | x | x |
| UV LIQ 116 | | | x | x | x | x | | |
| UV LIQ 117 | | | x | x | x | | x | |
| UV LIQ 118 | | | x | x | x | | | x |
| UV LIQ 119 | | | x | | x | x | | x |
| UV LIQ 120 | | | x | | | x | x | x |
| UV LIQ 121 | | | | x | x | x | x | |
| UV LIQ 122 | | | | x | x | x | | x |
| UV LIQ 123 | | | | | x | x | x | x |

Table 3 abbreviations

| Abbreviation | INCI name | Cas. No. |
|---|---|---|
| DHHB | Diethylamino Hydroxy Benzoyl Hexyl Benzoate | 302776-68-7 |
| EHDP | Ethylhexyl Dimethyl PABA | 21245-02-3 |
| EHMC | Ethylhexyl Methoxycinnamate | 5466-77-3 |
| EHS | Ethylhexyl Salicylate | 118-60-5 |
| HMS | Homosalate | 118-56-9 |
| IMC | Isoamyl p-Methoxycinnamate | 71617-10-2 |
| OCR | Octocrylene | 6197-30-4 |
| PS15 | Polysilicone-15 | 207574-74-1 |

The following mixtures of aqueous soluble or dispersible UV filters (Table 4) can be mixed together with the benzylidene malonates according to the present invention:

TABLE 4
List of aqueous soluble or dispersible UV filters

| | Abbreviation | | | | | | | | | | | | |
|---|---|---|---|---|---|---|---|---|---|---|---|---|---|
| | | | | | | | p- | | | | | | |
| Comb. Nr | BP5 | BCSA | CBM | DPDT | MBBT | PABA | PABA | PBSA | TDSA | TiO2 | ZnO | TBT | DHHM |
| UV WAT 1 | x | | | | | | | | | | | | |
| UV WAT 2 | | x | | | | | | | | | | | |
| UV WAT 3 | | | x | | | | | | | | | | |
| UV WAT 4 | | | | x | | | | | | | | | |
| UV WAT 5 | | | | | x | | | | | | | | |
| UV WAT 6 | | | | | | x | | | | | | | |
| UV WAT 7 | | | | | | | x | | | | | | |
| UV WAT 8 | | | | | | | | x | | | | | |
| UV WAT 9 | | | | | | | | | x | | | | |
| UV WAT 10 | | | | | | | | | | x | | | |
| UV WAT 11 | | | | | | | | | | | x | | |
| UV WAT 12 | | | | | | | | | | | | x | |
| UV WAT 13 | | | | | | | | | | | | | x |
| UV WAT 14 | x | x | | | | | | | | | | | |
| UV WAT 15 | x | | x | | | | | | | | | | |
| UV WAT 16 | x | | | x | | | | | | | | | |
| UV WAT 17 | x | | | | x | | | | | | | | |
| UV WAT 18 | x | | | | | x | | | | | | | |
| UV WAT 19 | x | | | | | | x | | | | | | |
| UV WAT 20 | x | | | | | | | x | | | | | |
| UV WAT 21 | x | | | | | | | | x | | | | |
| UV WAT 22 | x | | | | | | | | | x | | | |
| UV WAT 23 | x | | | | | | | | | | x | | |
| UV WAT 24 | x | | | | | | | | | | | x | |
| UV WAT 25 | x | | | | | | | | | | | | x |
| UV WAT 26 | | x | x | | | | | | | | | | |
| UV WAT 27 | | x | | x | | | | | | | | | |
| UV WAT 28 | | x | | | x | | | | | | | | |
| UV WAT 29 | | x | | | | x | | | | | | | |
| UV WAT 30 | | x | | | | | x | | | | | | |
| UV WAT 31 | | x | | | | | | x | | | | | |
| UV WAT 32 | | x | | | | | | | x | | | | |
| UV WAT 33 | | x | | | | | | | | x | | | |
| UV WAT 34 | | x | | | | | | | | | x | | |
| UV WAT 35 | | x | | | | | | | | | | x | |
| UV WAT 36 | | x | | | | | | | | | | | x |
| UV WAT 37 | | | x | x | | | | | | | | | |
| UV WAT 38 | | | x | | x | | | | | | | | |
| UV WAT 39 | | | x | | | x | | | | | | | |
| UV WAT 40 | | | x | | | | x | | | | | | |
| UV WAT 41 | | | x | | | | | x | | | | | |
| UV WAT 42 | | | x | | | | | | x | | | | |
| UV WAT 43 | | | x | | | | | | | x | | | |
| UV WAT 44 | | | x | | | | | | | | x | | |
| UV WAT 45 | | | x | | | | | | | | | x | |
| UV WAT 46 | | | x | | | | | | | | | | x |
| UV WAT 47 | | | | x | x | | | | | | | | |
| UV WAT 48 | | | | x | | x | | | | | | | |
| UV WAT 49 | | | | x | | | x | | | | | | |
| UV WAT 50 | | | | x | | | | x | | | | | |
| UV WAT 51 | | | | x | | | | | x | | | | |
| UV WAT 52 | | | | x | | | | | | x | | | |
| UV WAT 53 | | | | x | | | | | | | x | | |
| UV WAT 54 | | | | x | | | | | | | | x | |
| UV WAT 55 | | | | x | | | | | | | | | x |
| UV WAT 56 | | | | | x | x | | | | | | | |
| UV WAT 57 | | | | | x | | x | | | | | | |
| UV WAT 58 | | | | | x | | | x | | | | | |
| UV WAT 59 | | | | | x | | | | x | | | | |
| UV WAT 60 | | | | | x | | | | | x | | | |
| UV WAT 61 | | | | | x | | | | | | x | | |
| UV WAT 62 | | | | | x | | | | | | | x | |
| UV WAT 63 | | | | | x | | | | | | | | x |
| UV WAT 64 | | | | | | x | x | | | | | | |
| UV WAT 65 | | | | | | x | | x | | | | | |
| UV WAT 66 | | | | | | x | | | x | | | | |
| UV WAT 67 | | | | | | x | | | | x | | | |
| UV WAT 68 | | | | | | x | | | | | x | | |
| UV WAT 69 | | | | | | x | | | | | | x | |
| UV WAT 70 | | | | | | x | | | | | | | x |
| UV WAT 71 | | | | | | | x | x | | | | | |
| UV WAT 72 | | | | | | | x | | x | | | | |
| UV WAT 73 | | | | | | | x | | | x | | | |
| UV WAT 74 | | | | | | | x | | | | x | | |
| UV WAT 75 | | | | | | | x | | | | | x | |
| UV WAT 76 | | | | | | | x | | | | | | x |
| UV WAT 77 | | | | | | | | x | x | | | | |
| UV WAT 78 | | | | | | | | x | | x | | | |
| UV WAT 79 | | | | | | | | x | | | x | | |
| UV WAT 80 | | | | | | | | x | | | | x | |
| UV WAT 81 | | | | | | | | x | | | | | x |
| UV WAT 82 | | | | | | | | | x | x | | | |
| UV WAT 83 | | | | | | | | | x | | x | | |
| UV WAT 84 | | | | | | | | | x | | | x | |
| UV WAT 85 | | | | | | | | | x | | | | x |
| UV WAT 86 | | | | | | | | | | x | x | | |
| UV WAT 87 | | | | | | | | | | x | | x | |
| UV WAT 88 | | | | | | | | | | x | | | x |
| UV WAT 89 | | | | | | | | | | | x | x | |
| UV WAT 90 | | | | | | | | | | | x | | x |
| UV WAT 91 | | | | | | | | | | | | x | x |
| UV WAT 92 | x | x | x | | | | | | | | | | |
| UV WAT 93 | x | x | | x | | | | | | | | | |
| UV WAT 94 | x | x | | | x | | | | | | | | |
| UV WAT 95 | x | x | | | | x | | | | | | | |
| UV WAT 96 | x | x | | | | | x | | | | | | |
| UV WAT 97 | x | x | | | | | | x | | | | | |
| UV WAT 98 | x | x | | | | | | | x | | | | |
| UV WAT 99 | x | x | | | | | | | | x | | | |
| UV WAT 100 | x | x | | | | | | | | | x | | |
| UV WAT 101 | x | x | | | | | | | | | | x | |
| UV WAT 102 | x | x | | | | | | | | | | | x |
| UV WAT 103 | x | | x | x | | | | | | | | | |
| UV WAT 104 | x | | x | | x | | | | | | | | |
| UV WAT 105 | x | | x | | | x | | | | | | | |
| UV WAT 106 | x | | x | | | | x | | | | | | |
| UV WAT 107 | x | | x | | | | | x | | | | | |
| UV WAT 108 | x | | x | | | | | | x | | | | |
| UV WAT 109 | x | | x | | | | | | | x | | | |
| UV WAT 110 | x | | x | | | | | | | | x | | |
| UV WAT 111 | x | | x | | | | | | | | | x | |
| UV WAT 112 | x | | x | | | | | | | | | | x |
| UV WAT 113 | x | | | x | x | | | | | | | | |
| UV WAT 114 | x | | | x | | x | | | | | | | |
| UV WAT 115 | x | | | x | | | x | | | | | | |
| UV WAT 116 | x | | | x | | | | x | | | | | |
| UV WAT 117 | x | | | x | | | | | x | | | | |
| UV WAT 118 | x | | | x | | | | | | x | | | |
| UV WAT 119 | x | | | x | | | | | | | x | | |
| UV WAT 120 | x | | | x | | | | | | | | x | |
| UV WAT 121 | x | | | x | | | | | | | | | x |
| UV WAT 122 | x | | | | x | x | | | | | | | |
| UV WAT 123 | x | | | | x | | x | | | | | | |
| UV WAT 124 | x | | | | x | | | x | | | | | |
| UV WAT 125 | x | | | | x | | | | x | | | | |
| UV WAT 126 | x | | | | x | | | | | x | | | |
| UV WAT 127 | x | | | | x | | | | | | x | | |
| UV WAT 128 | x | | | | x | | | | | | | x | |
| UV WAT 129 | x | | | | x | | | | | | | | x |
| UV WAT 130 | x | | | | | x | x | | | | | | |
| UV WAT 131 | x | | | | | x | | x | | | | | |
| UV WAT 132 | x | | | | | x | | | x | | | | |
| UV WAT 133 | x | | | | | x | | | | x | | | |
| UV WAT 134 | x | | | | | x | | | | | x | | |
| UV WAT 135 | x | | | | | x | | | | | | x | |
| UV WAT 136 | x | | | | | x | | | | | | | x |
| UV WAT 137 | x | | | | | | x | x | | | | | |
| UV WAT 138 | x | | | | | | x | | x | | | | |
| UV WAT 139 | x | | | | | | x | | | x | | | |
| UV WAT 140 | x | | | | | | x | | | | x | | |
| UV WAT 141 | x | | | | | | x | | | | | x | |
| UV WAT 142 | x | | | | | | x | | | | | | x |
| UV WAT 143 | x | | | | | | | x | x | | | | |
| UV WAT 144 | x | | | | | | | x | | x | | | |
| UV WAT 145 | x | | | | | | | x | | | x | | |
| UV WAT 146 | x | | | | | | | x | | | | x | |
| UV WAT 147 | x | | | | | | | x | | | | | x |
| UV WAT 148 | x | | | | | | | | x | x | | | |
| UV WAT 149 | x | | | | | | | | x | | x | | |
| UV WAT 150 | x | | | | | | | | x | | | x | |
| UV WAT 151 | x | | | | | | | | x | | | | x |
| UV WAT 152 | x | | | | | | | | | x | x | | |
| UV WAT 153 | x | | | | | | | | | x | | x | |
| UV WAT 154 | x | | | | | | | | | x | | | x |
| UV WAT 155 | x | | | | | | | | | | x | x | |
| UV WAT 156 | x | | | | | | | | | | x | | x |
| UV WAT 157 | x | | | | | | | | | | | x | x |
| UV WAT 158 | | x | x | x | | | | | | | | | |
| UV WAT 159 | | x | x | | x | | | | | | | | |
| UV WAT 160 | | x | x | | | x | | | | | | | |
| UV WAT 161 | | x | x | | | | x | | | | | | |
| UV WAT 162 | | x | x | | | | | x | | | | | |
| UV WAT 163 | | x | x | | | | | | x | | | | |
| UV WAT 164 | | x | x | | | | | | | x | | | |
| UV WAT 165 | | x | x | | | | | | | | x | | |
| UV WAT 166 | | x | x | | | | | | | | | x | |
| UV WAT 167 | | x | x | | | | | | | | | | x |
| UV WAT 168 | | x | | x | x | | | | | | | | |
| UV WAT 169 | | x | | x | | x | | | | | | | |
| UV WAT 170 | | x | | x | | | x | | | | | | |
| UV WAT 171 | | x | | x | | | | x | | | | | |
| UV WAT 172 | | x | | x | | | | | x | | | | |
| UV WAT 173 | | x | | x | | | | | | x | | | |
| UV WAT 174 | | x | | x | | | | | | | x | | |
| UV WAT 175 | | x | | x | | | | | | | | x | |
| UV WAT 176 | | x | | x | | | | | | | | | x |
| UV WAT 177 | | x | | | x | x | | | | | | | |
| UV WAT 178 | | x | | | x | | x | | | | | | |
| UV WAT 179 | | x | | | x | | | x | | | | | |
| UV WAT 180 | | x | | | x | | | | x | | | | |
| UV WAT 181 | | x | | | x | | | | | x | | | |
| UV WAT 182 | | x | | | x | | | | | | x | | |
| UV WAT 183 | | x | | | x | | | | | | | x | |
| UV WAT 184 | | x | | | x | | | | | | | | x |
| UV WAT 185 | | x | | | | x | x | | | | | | |
| UV WAT 186 | | x | | | | x | | x | | | | | |
| UV WAT 187 | | x | | | | x | | | x | | | | |
| UV WAT 188 | | x | | | | x | | | | x | | | |
| UV WAT 189 | | x | | | | x | | | | | x | | |
| UV WAT 190 | | x | | | | x | | | | | | x | |
| UV WAT 191 | | x | | | | x | | | | | | | x |
| UV WAT 192 | | x | | | | | x | x | | | | | |
| UV WAT 193 | | x | | | | | x | | x | | | | |
| UV WAT 194 | | x | | | | | x | | | x | | | |
| UV WAT 195 | | x | | | | | x | | | | x | | |
| UV WAT 196 | | x | | | | | x | | | | | x | |
| UV WAT 197 | | x | | | | | x | | | | | | x |
| UV WAT 198 | | x | | | | | | x | x | | | | |
| UV WAT 199 | | x | | | | | | x | | x | | | |
| UV WAT 200 | | x | | | | | | x | | | x | | |
| UV WAT 201 | | x | | | | | | x | | | | x | |
| UV WAT 202 | | x | | | | | | x | | | | | x |
| UV WAT 203 | | x | | | | | | | x | x | | | |
| UV WAT 204 | | x | | | | | | | x | | x | | |
| UV WAT 205 | | x | | | | | | | x | | | x | |
| UV WAT 206 | | x | | | | | | | x | | | | x |
| UV WAT 207 | | x | | | | | | | | x | x | | |
| UV WAT 208 | | x | | | | | | | | x | | x | |
| UV WAT 209 | | x | | | | | | | | x | | | x |
| UV WAT 210 | | x | | | | | | | | | x | x | |
| UV WAT 211 | | x | | | | | | | | | x | | x |
| UV WAT 212 | | x | | | | | | | | | | x | x |
| UV WAT 213 | | | x | x | x | | | | | | | | |
| UV WAT 214 | | | x | x | | x | | | | | | | |
| UV WAT 215 | | | x | x | | | x | | | | | | |
| UV WAT 216 | | | x | x | | | | x | | | | | |
| UV WAT 217 | | | x | x | | | | | x | | | | |
| UV WAT 218 | | | x | x | | | | | | x | | | |
| UV WAT 219 | | | x | x | | | | | | | x | | |
| UV WAT 220 | | | x | x | | | | | | | | x | |
| UV WAT 221 | | | x | x | | | | | | | | | x |
| UV WAT 222 | | | x | | x | x | | | | | | | |
| UV WAT 223 | | | x | | x | | x | | | | | | |
| UV WAT 224 | | | x | | x | | | x | | | | | |
| UV WAT 225 | | | x | | x | | | | x | | | | |
| UV WAT 226 | | | x | | x | | | | | x | | | |
| UV WAT 227 | | | x | | x | | | | | | x | | |
| UV WAT 228 | | | x | | x | | | | | | | x | |
| UV WAT 229 | | | x | | x | | | | | | | | x |
| UV WAT 230 | | | x | | | x | x | | | | | | |
| UV WAT 231 | | | x | | | x | | x | | | | | |
| UV WAT 232 | | | x | | | x | | | x | | | | |
| UV WAT 233 | | | x | | | x | | | | x | | | |
| UV WAT 234 | | | x | | | x | | | | | x | | |
| UV WAT 235 | | | x | | | x | | | | | | x | |
| UV WAT 236 | | | x | | | x | | | | | | | x |
| UV WAT 237 | | | x | | | | x | x | | | | | |
| UV WAT 238 | | | x | | | | x | | x | | | | |
| UV WAT 239 | | | x | | | | x | | | x | | | |
| UV WAT 240 | | | x | | | | x | | | | x | | |
| UV WAT 241 | | | x | | | | x | | | | | x | |
| UV WAT 242 | | | x | | | | x | | | | | | x |
| UV WAT 243 | | | x | | | | | x | x | | | | |
| UV WAT 244 | | | x | | | | | x | | x | | | |
| UV WAT 245 | | | x | | | | | x | | | x | | |
| UV WAT 246 | | | x | | | | | x | | | | x | |
| UV WAT 247 | | | x | | | | | x | | | | | x |
| UV WAT 248 | | | x | | | | | | x | x | | | |
| UV WAT 249 | | | x | | | | | | x | | x | | |
| UV WAT 250 | | | x | | | | | | x | | | x | |
| UV WAT 251 | | | x | | | | | | x | | | | x |
| UV WAT 252 | | | x | | | | | | | x | x | | |
| UV WAT 253 | | | x | | | | | | | x | | x | |
| UV WAT 254 | | | x | | | | | | | x | | | x |
| UV WAT 255 | | | x | | | | | | | | x | x | |
| UV WAT 256 | | | x | | | | | | | | x | | x |
| UV WAT 257 | | | x | | | | | | | | | x | x |
| UV WAT 258 | | | | x | x | x | | | | | | | |
| UV WAT 259 | | | | x | x | | x | | | | | | |
| UV WAT 260 | | | | x | x | | | x | | | | | |
| UV WAT 261 | | | | x | x | | | | x | | | | |
| UV WAT 262 | | | | x | x | | | | | x | | | |
| UV WAT 263 | | | | x | x | | | | | | x | | |
| UV WAT 264 | | | | x | x | | | | | | | x | |
| UV WAT 265 | | | | x | x | | | | | | | | x |
| UV WAT 266 | | | | x | | x | x | | | | | | |
| UV WAT 267 | | | | x | | x | | x | | | | | |
| UV WAT 268 | | | | x | | x | | | x | | | | |
| UV WAT 269 | | | | x | | x | | | | x | | | |
| UV WAT 270 | | | | x | | x | | | | | x | | |
| UV WAT 271 | | | | x | | x | | | | | | x | |
| UV WAT 272 | | | | x | | x | | | | | | | x |
| UV WAT 273 | | | | x | | | x | x | | | | | |
| UV WAT 274 | | | | x | | | x | | x | | | | |
| UV WAT 275 | | | | x | | | x | | | x | | | |
| UV WAT 276 | | | | x | | | x | | | | x | | |
| UV WAT 277 | | | | x | | | x | | | | | x | |
| UV WAT 278 | | | | x | | | x | | | | | | x |
| UV WAT 279 | | | | x | | | | x | x | | | | |
| UV WAT 280 | | | | x | | | | x | | x | | | |
| UV WAT 281 | | | | x | | | | x | | | x | | |
| UV WAT 282 | | | | x | | | | x | | | | x | |
| UV WAT 283 | | | | x | | | | x | | | | | x |
| UV WAT 284 | | | | x | | | | | x | x | | | |
| UV WAT 285 | | | | x | | | | | x | | x | | |
| UV WAT 286 | | | | x | | | | | x | | | x | |
| UV WAT 287 | | | | x | | | | | x | | | | x |
| UV WAT 288 | | | | x | | | | | | x | x | | |
| UV WAT 289 | | | | x | | | | | | x | | x | |
| UV WAT 290 | | | | x | | | | | | x | | | x |
| UV WAT 291 | | | | x | | | | | | | x | x | |
| UV WAT 292 | | | | x | | | | | | | x | | x |
| UV WAT 293 | | | | x | | | | | | | | x | x |
| UV WAT 294 | | | | | x | x | x | | | | | | |
| UV WAT 295 | | | | | x | x | | x | | | | | |
| UV WAT 296 | | | | | x | x | | | x | | | | |
| UV WAT 297 | | | | | x | x | | | | x | | | |
| UV WAT 298 | | | | | x | x | | | | | x | | |
| UV WAT 299 | | | | | x | x | | | | | | x | |
| UV WAT 300 | | | | | x | x | | | | | | | x |
| UV WAT 301 | | | | | x | | x | x | | | | | |
| UV WAT 302 | | | | | x | | x | | x | | | | |
| UV WAT 303 | | | | | x | | x | | | x | | | |
| UV WAT 304 | | | | | x | | x | | | | x | | |
| UV WAT 305 | | | | | x | | x | | | | | x | |
| UV WAT 306 | | | | | x | | x | | | | | | x |
| UV WAT 307 | | | | | x | | | x | x | | | | |
| UV WAT 308 | | | | | x | | | x | | x | | | |
| UV WAT 309 | | | | | x | | | x | | | x | | |
| UV WAT 310 | | | | | x | | | x | | | | x | |
| UV WAT 311 | | | | | x | | | x | | | | | x |
| UV WAT 312 | | | | | x | | | | x | x | | | |
| UV WAT 313 | | | | | x | | | | x | | x | | |
| UV WAT 314 | | | | | x | | | | x | | | x | |
| UV WAT 315 | | | | | x | | | | x | | | | x |
| UV WAT 316 | | | | | x | | | | | x | x | | |
| UV WAT 317 | | | | | x | | | | | x | | x | |
| UV WAT 318 | | | | | x | | | | | x | | | x |
| UV WAT 319 | | | | | x | | | | | | x | x | |
| UV WAT 320 | | | | | x | | | | | | x | | x |
| UV WAT 321 | | | | | x | | | | | | | x | x |
| UV WAT 322 | | | | | | x | x | x | | | | | |
| UV WAT 323 | | | | | | x | x | | x | | | | |
| UV WAT 324 | | | | | | x | x | | | x | | | |
| UV WAT 325 | | | | | | x | x | | | | x | | |
| UV WAT 326 | | | | | | x | x | | | | | x | |
| UV WAT 327 | | | | | | x | x | | | | | | x |
| UV WAT 328 | | | | | | x | | x | x | | | | |
| UV WAT 329 | | | | | | x | | x | | x | | | |
| UV WAT 330 | | | | | | x | | x | | | x | | |
| UV WAT 331 | | | | | | x | | x | | | | x | |
| UV WAT 332 | | | | | | x | | x | | | | | x |
| UV WAT 333 | | | | | | x | | | x | x | | | |
| UV WAT 334 | | | | | | x | | | x | | x | | |
| UV WAT 335 | | | | | | x | | | x | | | x | |
| UV WAT 336 | | | | | | x | | | x | | | | x |
| UV WAT 337 | | | | | | x | | | | x | x | | |
| UV WAT 338 | | | | | | x | | | | x | | x | |
| UV WAT 339 | | | | | | x | | | | x | | | x |
| UV WAT 340 | | | | | | x | | | | | x | x | |
| UV WAT 341 | | | | | | x | | | | | x | | x |
| UV WAT 342 | | | | | | x | | | | | | x | x |
| UV WAT 343 | | | | | | | x | x | x | | | | |
| UV WAT 344 | | | | | | | x | x | | x | | | |
| UV WAT 345 | | | | | | | x | x | | | x | | |
| UV WAT 346 | | | | | | | x | x | | | | x | |
| UV WAT 347 | | | | | | | x | x | | | | | x |
| UV WAT 348 | | | | | | | x | | x | x | | | |
| UV WAT 349 | | | | | | | x | | x | | x | | |
| UV WAT 350 | | | | | | | x | | x | | | x | |
| UV WAT 351 | | | | | | | x | | x | | | | x |
| UV WAT 352 | | | | | | | x | | | x | x | | |
| UV WAT 353 | | | | | | | x | | | x | | x | |
| UV WAT 354 | | | | | | | x | | | x | | | x |
| UV WAT 355 | | | | | | | x | | | | x | x | |
| UV WAT 356 | | | | | | | x | | | | x | | x |
| UV WAT 357 | | | | | | | x | | | | | x | x |
| UV WAT 358 | | | | | | | | x | x | x | | | |
| UV WAT 359 | | | | | | | | x | x | | x | | |
| UV WAT 360 | | | | | | | | x | x | | | x | |
| UV WAT 361 | | | | | | | | x | x | | | | x |
| UV WAT 362 | | | | | | | | x | | x | x | | |
| UV WAT 363 | | | | | | | | x | | x | | x | |
| UV WAT 364 | | | | | | | | x | | x | | | x |
| UV WAT 365 | | | | | | | | x | | | x | x | |
| UV WAT 366 | | | | | | | | x | | | x | | x |
| UV WAT 367 | | | | | | | | x | | | | x | x |
| UV WAT 368 | | | | | | | | | x | x | x | | |
| UV WAT 369 | | | | | | | | | x | x | | x | |
| UV WAT 370 | | | | | | | | | x | x | | | x |
| UV WAT 371 | | | | | | | | | x | | x | x | |
| UV WAT 372 | | | | | | | | | x | | x | | x |
| UV WAT 373 | | | | | | | | | x | | | x | x |
| UV WAT 374 | | | | | | | | | | x | x | x | |
| UV WAT 375 | | | | | | | | | | x | x | | x |
| UV WAT 376 | | | | | | | | | | x | | x | x |
| UV WAT 377 | | | | | | | | | | | x | x | x |

Table 4 Abbreviations

| | | Particle size | |
|---|---|---|---|
| Abbreviations | INCI name | range | Cas. No. |
| BP5 | Benzophenone-5 | | 6628-37-1 |
| BCSA | Benzydilene Camphor Sulfonic Acid | | 56039-58-8 |
| CBM | Camphor Benzalkonium Methosulfate | | 52793-97-2 |
| DPDT | Disodium Phenyl Dibenzylmidazole | | 180898-37-7 |
| | Tetrasulfonate | | |
| MBBT | Micronized Methylene Bis-Benzotriazolyl | 50-200 nm | 103597-45-1 |
| | Tatramethylbutylphenol | | |
| PABA | PABA | | 150-13-0 |
| p-PABA | PEG-25 PABA | | 113010-52-9 |
| PBSA | Phenylbenzimidazole Sulfonic Acid | | 27503-81-7 |
| TDSA | Terephthalylidene Dicamphor Sulfonic Acid | | 90457-82-2 |
| TiO₂ | Titanium Dioxide | 10-50 nm | 13463-67-7 |
| ZnO | Zinc Oxide | 20-100 nm | 131413-2 |
| TB | Micronized Tris-Biphenyl Triazine | 50-200 nm | 31274-51-8 |
| DHHM | Micronized (2-{4-[2-(4-Diethylamino-2-hydroxy- | 50-200 nm | 919803-06-8 |
| | benzoyl)-benzoyl]-piperazine-1-carbonyl}- | | |
| | phenyl)-(4-diethylamino-2-hydroxy-phenyl)- | | |
| | methanone | | |

### UV FILTER COMBINATION EXAMPLES

In all of the UV filter combinations listed before in the Table “X” represents a specific UV filter. The weight ratio of each specific UV absorber (based on the weight of all UV absorbers in the combination) can for example range from 0.01 to 0.99, especially 0.1 to 0.9, preferably 0.2 to 0.8. (for example 0.3).

Further UV filter combination examples are the following:

In all of the UV filter combinations listed before the weight ratio of the UV absorbers (based on the weight of all UV absorbers in the combination) is:

(a) for combinations of two UV absorbers:

1:1 or 1:2 or 2:1 or 1:3 or 3:1.

(b) for combinations of three UV absorbers:

1:1:1 or 1:2:1 or 1:1:2 or 2:1:1 or 1:2:2 or 2:1:2 or 2:2:1 or 1:3:1 or 1:1:3 or 3:1:1 or 1:3:3 or 3:1:3 or 3:3:1 or 1:2:3 or 1:3:2 or 2:1:3 or 2:3:1 or 3:1:2 or 3:2:1.

(c) for combinations of four UV absorbers:

1:1:1:1 or 1:1:2:1 or 1:1:1:2 or 1:2:1:1 or 2:1:1:1 or 1:1:1:3 or 1:1:3:1

or 1:3:1:1 or 3:1:1:1 or 1:2:2:1 or 2:1:2:1 or 2:2:1:1 or 2:1:1:2 or

1:3:31 or 3:1:3:1 or 3:3:1:1 or 3:1:1:3 or 1:2:3:1 or 1:3:2:1 or 1:1:2:3

or 1:1:3:2 or 2:1:1:3 or 2:1:3:1 or 2:3:1:1 or 3:1:1.2 or 3:2:1:1 or

3:1:2:1.

### FORMULATION EXAMPLES

In the following formulation examples:

The new UV filter may be (as described in Table 1) DBM-01 or DBM-02 or DBM-03 or DBM-04 or DBM-05 or DBM-06 or DBM-07 or DBM-081 or DBM-09 or DBM-10 or DBM-11 or DBM-12 or DBM-13 or DBM-14 or DBM-15 or DBM-16 or DBM-17 or DBM-18 or DBM-19 or DBM-20 or DBM-21.

“UV SOL” may be (as described in Table 2) UV SOL 1, or UV SOL 2, or UV SOL 3, or UV SOL 4, or UV SOL 5, or UV SOL 6, or UV SOL 7, or UV SOL 8, or UV SOL 9, or UV SOL 10, or UV SOL 11, or UV SOL 12, or UV SOL 13, or UV SOL 14, or UV SOL 15, or UV SOL 16, or UV SOL 17, or UV SOL 18, or UV SOL 19, or UV SOL 20, or UV SOL 21, or UV SOL 22, or UV SOL 23, or UV SOL 24, or UV SOL 25, or UV SOL 26, or UV SOL 27, or UV SOL 28, or UV SOL 29, or UV SOL 30, or UV SOL 31, or UV SOL 32, or UV SOL 33, or UV SOL 34, or UV SOL 35, or UV SOL 36, or UV SOL 37, or UV SOL 38, or UV SOL 39, or UV SOL 40, or UV SOL 41, or UV SOL 42, or UV SOL 43, or UV SOL 44, or UV SOL 45, or UV SOL 46, or UV SOL 47, or UV SOL 48, or UV SOL 49, or UV SOL 50, or UV SOL 51, or UV SOL 52, or UV SOL 53, or UV SOL 54, or UV SOL 55, or UV SOL 56, or UV SOL 57, or UV SOL 58, or UV SOL 59, or UV SOL 60, or UV SOL 61, or UV SOL 62, or UV SOL 63, or UV SOL 64, or UV SOL 65, or UV SOL 66, or UV SOL 67, or UV SOL 68, or UV SOL 69, or UV SOL 70, or UV SOL 71, or UV SOL 72, or UV SOL 73, or UV SOL 74, or UV SOL 75, or UV SOL 76, or UV SOL 77, or UV SOL 78, or UV SOL 79, or UV SOL 80, or UV SOL 81, or UV SOL 82, or UV SOL 83, or UV SOL 84, or UV SOL 85, or UV SOL 86, or UV SOL 87, or UV SOL 88, or UV SOL 89, or UV SOL 90, or UV SOL 91, or UV SOL 92, or UV SOL 93, or UV SOL 94, or UV SOL 95, or UV SOL 96, or UV SOL 97, or UV SOL 98, or UV SOL 99, or UV SOL 100, or UV SOL 101, or UV SOL 102, or UV SOL 103, or UV SOL 104, or UV SOL 105, or UV SOL 106, or UV SOL 107, or UV SOL 108, or UV SOL 109, or UV SOL 110, or UV SOL 111, or UV SOL 112, or UV SOL 113, or UV SOL 114, or UV SOL 115, or UV SOL 116, or UV SOL 117, or UV SOL 118, or UV SOL 119, or UV SOL 120, or UV SOL 121, or UV SOL 122, or UV SOL 123, or UV SOL 124, or UV SOL 125, or UV SOL 126, or UV SOL 127, or UV SOL 128, or UV SOL 129, or UV SOL 130, or UV SOL 131, or UV SOL 132, or UV SOL 133, or UV SOL 134, or UV SOL 135, or UV SOL 136, or UV SOL 137, or UV SOL 138, or UV SOL 139, or UV SOL 140, or UV SOL 141, or UV SOL 142, or UV SOL 143, or UV SOL 144, or UV SOL 145, or UV SOL 146, or UV SOL 147, or UV SOL 148, or UV SOL 149, or UV SOL 150, or UV SOL 151, or UV SOL 152, or UV SOL 153, or UV SOL 154, or UV SOL 155, or UV SOL 156, or UV SOL 157, or UV SOL 158, or UV SOL 159, or UV SOL 160, or UV SOL 161, or UV SOL 162, or UV SOL 163, or UV SOL 164, or UV SOL 165, or UV SOL 166, or UV SOL 167, or UV SOL 168, or UV SOL 169, or UV SOL 170, or UV SOL 171, or UV SOL 172, or UV SOL 173, or UV SOL 174, or UV SOL 175, or UV SOL 176, or UV SOL 177, or UV SOL 178, or UV SOL 179, or UV SOL 180, or UV SOL 181, or UV SOL 182, or UV SOL 183, or UV SOL 184, or UV SOL 185, or UV SOL 186, or UV SOL 187, or UV SOL 188, or UV SOL 189, or UV SOL 190, or UV SOL 191, or UV SOL 192, or UV SOL 193, or UV SOL 194, or UV SOL 195, or UV SOL 196, or UV SOL 197, or UV SOL 198, or UV SOL 199, or UV SOL 200, or UV SOL 201, or UV SOL 202, or UV SOL 203, or UV SOL 204, or UV SOL 205, or UV SOL 206, or UV SOL 207, or UV SOL 208, or UV SOL 209, or UV SOL 210, or UV SOL 211, or UV SOL 212, or UV SOL 213, or UV SOL 214, or UV SOL 215, or UV SOL 216, or UV SOL 217, or UV SOL 218, or UV SOL 219, or UV SOL 220, or UV SOL 221, or UV SOL 222, or UV SOL 223, or UV SOL 224, or UV SOL 225, or UV SOL 226, or UV SOL 227, or UV SOL 228, or UV SOL 229, or UV SOL 230, or UV SOL 231, or UV SOL 232, or UV SOL 233, or UV SOL 234, or UV SOL 235, or UV SOL 236, or UV SOL 237, or UV SOL 238, or UV SOL 239, or UV SOL 240, or UV SOL 241, or UV SOL 242, or UV SOL 243, or UV SOL 244, or UV SOL 245, or UV SOL 246, or UV SOL 247, or UV SOL 248, or UV SOL 249, or UV SOL 250, or UV SOL 251, or UV SOL 252, or UV SOL 253;

“UV LIQ” may be (as described in Table 3) UV LIQ 1, or UV LIQ 2, or UV LIQ 3, or UV LIQ 4, or UV LIQ 5, or UV LIQ 6, or UV LIQ 7, or UV LIQ 8, or UV LIQ 9, or UV LIQ 10, or UV LIQ 11, or UV LIQ 12, or UV LIQ 13, or UV LIQ 14, or UV LIQ 15, or UV LIQ 16, or UV LIQ 17, or UV LIQ 18, or UV LIQ 19, or UV LIQ 20, or UV LIQ 21, or UV LIQ 22, or UV LIQ 23, or UV LIQ 24, or UV LIQ 25, or UV LIQ 26, or UV LIQ 27, or UV LIQ 28, or UV LIQ 29, or UV LIQ 30, or UV LIQ 31, or UV LIQ 32, or UV LIQ 33, or UV LIQ 34, or UV LIQ 35, or UV LIQ 36, or UV LIQ 37, or UV LIQ 38, or UV LIQ 39, or UV LIQ 40, or UV LIQ 41, or UV LIQ 42, or UV LIQ 43, or UV LIQ 44, or UV LIQ 45, or UV LIQ 46, or UV LIQ 47, or UV LIQ 48, or UV LIQ 49, or UV LIQ 50, or UV LIQ 51, or UV LIQ 52, or UV LIQ 53, or UV LIQ 54, or UV LIQ 55, or UV LIQ 56, or UV LIQ 57, or UV LIQ 58, or UV LIQ 59, or UV LIQ 60, or UV LIQ 61, or UV LIQ 62, or UV LIQ 63, or UV LIQ 64, or UV LIQ 65, or UV LIQ 66, or UV LIQ 67, or UV LIQ 68, or UV LIQ 69, or UV LIQ 70, or UV LIQ 71, or UV LIQ 72, or UV LIQ 73, or UV LIQ 74, or UV LIQ 75, or UV LIQ 76, or UV LIQ 77, or UV LIQ 78, or UV LIQ 79, or UV LIQ 80, or UV LIQ 81, or UV LIQ 82, or UV LIQ 83, or UV LIQ 84, or UV LIQ 85, or UV LIQ 86, or UV LIQ 87, or UV LIQ 88, or UV LIQ 89, or UV LIQ 90, or UV LIQ 91, or UV LIQ 92, or UV LIQ 93, or UV LIQ 94, or UV LIQ 95, or UV LIQ 96, or UV LIQ 97, or UV LIQ 98, or UV LIQ 99, or UV LIQ 100, or UV LIQ 101, or UV LIQ 102, or UV LIQ 103, or UV LIQ 104, or UV LIQ 105, or UV LIQ 106, or UV LIQ 107, or UV LIQ 108, or UV LIQ 109, or UV LIQ 110, or UV LIQ 111, or UV LIQ 112, or UV LIQ 113, or UV LIQ 114, or UV LIQ 115, or UV LIQ 116, or UV LIQ 117, or UV LIQ 118, or UV LIQ 119, or UV LIQ 120, or UV LIQ 121, or UV LIQ 122, or UV LIQ 123;

“UV WAT” may be (as described in Table 4) UV WAT 1, or UV WAT 2, or UV WAT 3, or UV WAT 4, or UV WAT 5, or UV WAT 6, or UV WAT 7, or UV WAT 8, or UV WAT 9, or UV WAT 10, or UV WAT 11, or UV WAT 12, or UV WAT 13, or UV WAT 14, or UV WAT 15, or UV WAT 16, or UV WAT 17, or UV WAT 18, or UV WAT 19, or UV WAT 20, or UV WAT 21, or UV WAT 22, or UV WAT 23, or UV WAT 24, or UV WAT 25, or UV WAT 26, or UV WAT 27, or UV WAT 28, or UV WAT 29, or UV WAT 30, or UV WAT 31, or UV WAT 32, or UV WAT 33, or UV WAT 34, or UV WAT 35, or UV WAT 36, or UV WAT 37, or UV WAT 38, or UV WAT 39, or UV WAT 40, or UV WAT 41, or UV WAT 42, or UV WAT 43, or UV WAT 44, or UV WAT 45, or UV WAT 46, or UV WAT 47, or UV WAT 48, or UV WAT 49, or UV WAT 50, or UV WAT 51, or UV WAT 52, or UV WAT 53, or UV WAT 54, or UV WAT 55, or UV WAT 56, or UV WAT 57, or UV WAT 58, or UV WAT 59, or UV WAT 60, or UV WAT 61, or UV WAT 62, or UV WAT 63, or UV WAT 64, or UV WAT 65, or UV WAT 66, or UV WAT 67, or UV WAT 68, or UV WAT 69, or UV WAT 70, or UV WAT 71, or UV WAT 72, or UV WAT 73, or UV WAT 74, or UV WAT 75, or UV WAT 76, or UV WAT 77, or UV WAT 78, or UV WAT 79, or UV WAT 80, or UV WAT 81, or UV WAT 82, or UV WAT 83, or UV WAT 84, or UV WAT 85, or UV WAT 86, or UV WAT 87, or UV WAT 88, or UV WAT 89, or UV WAT 90, or UV WAT 91, or UV WAT 92, or UV WAT 93, or UV WAT 94, or UV WAT 95, or UV WAT 96, or UV WAT 97, or UV WAT 98, or UV WAT 99, or UV WAT 100, or UV WAT 101, or UV WAT 102, or UV WAT 103, or UV WAT 104, or UV WAT 105, or UV WAT 106, or UV WAT 107, or UV WAT 108, or UV WAT 109, or UV WAT 110, or UV WAT 111, or UV WAT 112, or UV WAT 113, or UV WAT 114, or UV WAT 115, or UV WAT 116, or UV WAT 117, or UV WAT 118, or UV WAT 119, or UV WAT 120, or UV WAT 121, or UV WAT 122, or UV WAT 123, or UV WAT 124, or UV WAT 125, or UV WAT 126, or UV WAT 127, or UV WAT 128, or UV WAT 129, or UV WAT 130, or UV WAT 131, or UV WAT 132, or UV WAT 133, or UV WAT 134, or UV WAT 135, or UV WAT 136, or UV WAT 137, or UV WAT 138, or UV WAT 139, or UV WAT 140, or UV WAT 141, or UV WAT 142, or UV WAT 143, or UV WAT 144, or UV WAT 145, or UV WAT 146, or UV WAT 147, or UV WAT 148, or UV WAT 149, or UV WAT 150, or UV WAT 151, or UV WAT 152, or UV WAT 153, or UV WAT 154, or UV WAT 155, or UV WAT 156, or UV WAT 157, or UV WAT 158, or UV WAT 159, or UV WAT 160, or UV WAT 161, or UV WAT 162, or UV WAT 163, or UV WAT 164, or UV WAT 165, or UV WAT 166, or UV WAT 167, or UV WAT 168, or UV WAT 169, or UV WAT 170, or UV WAT 171, or UV WAT 172, or UV WAT 173, or UV WAT 174, or UV WAT 175, or UV WAT 176, or UV WAT 177, or UV WAT 178, or UV WAT 179, or UV WAT 180, or UV WAT 181, or UV WAT 182, or UV WAT 183, or UV WAT 184, or UV WAT 185, or UV WAT 186, or UV WAT 187, or UV WAT 188, or UV WAT 189, or UV WAT 190, or UV WAT 191, or UV WAT 192, or UV WAT 193, or UV WAT 194, or UV WAT 195, or UV WAT 196, or UV WAT 197, or UV WAT 198, or UV WAT 199, or UV WAT 200, or UV WAT 201, or UV WAT 202, or UV WAT 203, or UV WAT 204, or UV WAT 205, or UV WAT 206, or UV WAT 207, or UV WAT 208, or UV WAT 209, or UV WAT 210, or UV WAT 211, or UV WAT 212, or UV WAT 213, or UV WAT 214, or UV WAT 215, or UV WAT 216, or UV WAT 217, or UV WAT 218, or UV WAT 219, or UV WAT 220, or UV WAT 221, or UV WAT 222, or UV WAT 223, or UV WAT 224, or UV WAT 225, or UV WAT 226, or UV WAT 227, or UV WAT 228, or UV WAT 229, or UV WAT 230, or UV WAT 231, or UV WAT 232, or UV WAT 233, or UV WAT 234, or UV WAT 235, or UV WAT 236, or UV WAT 237, or UV WAT 238, or UV WAT 239, or UV WAT 240, or UV WAT 241, or UV WAT 242, or UV WAT 243, or UV WAT 244, or UV WAT 245, or UV WAT 246, or UV WAT 247, or UV WAT 248, or UV WAT 249, or UV WAT 250, or UV WAT 251, or UV WAT 252, or UV WAT 253, or UV WAT 254, or UV WAT 255, or UV WAT 256, or UV WAT 257, or UV WAT 258, or UV WAT 259, or UV WAT 260, or UV WAT 261, or UV WAT 262, or UV WAT 263, or UV WAT 264, or UV WAT 265, or UV WAT 266, or UV WAT 267, or UV WAT 268, or UV WAT 269, or UV WAT 270, or UV WAT 271, or UV WAT 272, or UV WAT 273, or UV WAT 274, or UV WAT 275, or UV WAT 276, or UV WAT 277, or UV WAT 278, or UV WAT 279, or UV WAT 280, or UV WAT 281, or UV WAT 282, or UV WAT 283, or UV WAT 284, or UV WAT 285, or UV WAT 286, or UV WAT 287, or UV WAT 288, or UV WAT 289, or UV WAT 290, or UV WAT 291, or UV WAT 292, or UV WAT 293, or UV WAT 294, or UV WAT 295, or UV WAT 296, or UV WAT 297, or UV WAT 298, or UV WAT 299, or UV WAT 300, or UV WAT 301, or UV WAT 302, or UV WAT 303, or UV WAT 304, or UV WAT 305, or UV WAT 306, or UV WAT 307, or UV WAT 308, or UV WAT 309, or UV WAT 310, or UV WAT 311, or UV WAT 312, or UV WAT 313, or UV WAT 314, or UV WAT 315, or UV WAT 316, or UV WAT 317, or UV WAT 318, or UV WAT 319, or UV WAT 320, or UV WAT 321, or UV WAT 322, or UV WAT 323, or UV WAT 324, or UV WAT 325, or UV WAT 326, or UV WAT 327, or UV WAT 328, or UV WAT 329, or UV WAT 330, or UV WAT 331, or UV WAT 332, or UV WAT 333, or UV WAT 334, or UV WAT 335, or UV WAT 336, or UV WAT 337, or UV WAT 338, or UV WAT 339, or UV WAT 340, or UV WAT 341, or UV WAT 342, or UV WAT 343, or UV WAT 344, or UV WAT 345, or UV WAT 346, or UV WAT 347, or UV WAT 348, or UV WAT 349, or UV WAT 350, or UV WAT 351, or UV WAT 352, or UV WAT 353, or UV WAT 354, or UV WAT 355, or UV WAT 356, or UV WAT 357, or UV WAT 358, or UV WAT 359, or UV WAT 360, or UV WAT 361, or UV WAT 362, or UV WAT 363, or UV WAT 364, or UV WAT 365, or UV WAT 366, or UV WAT 367, or UV WAT 368, or UV WAT 369, or UV WAT 370, or UV WAT 371, or UV WAT 372, or UV WAT 373, or UV WAT 374, or UV WAT 375, or UV WAT 376, or UV WAT 377.

### FORMULATION EXAMPLES

| | | Emulsion high Protection | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Cyclomethicone | 7.0 | 7.0 | 7.0 | 7.0 | 7.0 | 7.0 | 7.0 | 7.0 | 7.0 |
| | Ethylhexyl Palmitate | 5.0 | 0.0 | 0.0 | 0.0 | 0.0 | 0.0 | 0.0 | 0.0 | 0.0 |
| | New UV filter | | 3.0 | 15.0 | 5.0 | 10.0 | 5.0 | 8.0 | 5.0 | 15.0 |
| | Glyceryl Stearate | 1.50 | 1.50 | 1.50 | 1.50 | 1.50 | 1.50 | 1.50 | 1.50 | 1.50 |
| | Potassium Cetyl | 1.80 | 1.80 | 1.80 | 1.80 | 1.80 | 1.80 | 1.80 | 1.80 | 1.80 |
| | Phosphate | | | | | | | | | |
| | VP/Eicosene Copolymer | 0.60 | 0.60 | 0.60 | 0.60 | 0.60 | 0.60 | 0.60 | 0.60 | 0.60 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part B | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Acrylates/Palmeth-25 | 0.25 | 0.25 | 0.25 | 0.25 | 0.25 | 0.25 | 0.25 | 0.25 | 0.25 |
| | Acrylate Copolymer | | | | | | | | | |
| | Glycerin | 6.0 | 6.0 | 6.0 | 6.0 | 6.0 | 6.0 | 6.0 | 6.0 | 6.0 |
| | Disodium EDTA | 0.20 | 0.20 | 0.20 | 0.20 | 0.20 | 0.20 | 0.20 | 0.20 | 0.20 |
| | UV WAT | | 5.0 | 5.0 | 5.0 | 5.0 | 5.0 | 5.0 | 0.0 | 5.0 |
| Part C | Phenoxyethanol (and) | 0.70 | 0.70 | 0.70 | 0.70 | 0.70 | 0.70 | 0.70 | 0.70 | 0.70 |
| | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| | Tocopheryl Acetate | 0.30 | 0.30 | 0.30 | 0.30 | 0.30 | 0.30 | 0.30 | 0.30 | 0.30 |

| | | Sun Cream | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Cetearyl glucoside | 4.0 | 4.0 | 4.0 | 4.0 | 4.0 | 4.0 | 4.0 | 4.0 | 4.0 |
| | Dicaprylyl Carbonate | 8.0 | 0.0 | 0.0 | 0.0 | 0.0 | 0.0 | 0.0 | 0.0 | 0.0 |
| | New UV filter | | 3.0 | 15.0 | 5.0 | 10.0 | 5.0 | 8.0 | 5.0 | 15.0 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part B | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Disodium EDTA | 0.20 | 0.20 | 0.20 | 0.20 | 0.20 | 0.20 | 0.20 | 0.20 | 0.20 |
| | Glycerin | 6.0 | 6.0 | 6.0 | 6.0 | 6.0 | 6.0 | 6.0 | 6.0 | 6.0 |
| | PVP/dimethylconylacrylate/ | 3.0 | 3.0 | 3.0 | 3.0 | 3.0 | 3.0 | 3.0 | 3.0 | 3.0 |
| | polycarbamyl/polyglycol | | | | | | | | | |
| | ester | | | | | | | | | |
| | UV WAT | | 5.0 | 5.0 | 5.0 | 5.0 | 5.0 | 5.0 | 0.0 | 5.0 |
| | Sodium polyacrylate | 0.70 | 0.70 | 0.70 | 0.70 | 0.70 | 0.70 | 0.70 | 0.70 | 0.70 |
| Part C | Dimethicone | 3.0 | 3.0 | 3.0 | 3.0 | 3.0 | 3.0 | 3.0 | 3.0 | 3.0 |
| | Corn Starch modified | 3.0 | 3.0 | 3.0 | 3.0 | 3.0 | 3.0 | 3.0 | 3.0 | 3.0 |
| Part E | Phenoxyethanol (and) | 1.0 | 1.0 | 1.0 | 1.0 | 1.0 | 1.0 | 1.0 | 1.0 | 1.0 |
| | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |

| | Sunscreen Gel | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Alcohol Denatured | Qs to | Qs to | Qs to | Qs to | Qs to | Qs to | Qs to | Qs to | Qs to |
| | 100 | 100 | 100 | 100 | 100 | 100 | 100 | 100 | 100 |
| Hydroxypropyl Cellulose | 1.0 | 1.0 | 1.0 | 1.0 | 1.0 | 1.0 | 1.0 | 1.0 | 1.0 |
| Acrylates/Octylacrylamide | 1.0 | 1.0 | 1.0 | 1.0 | 1.0 | 1.0 | 1.0 | 1.0 | 1.0 |
| Copolymer | | | | | | | | | |
| C12-15 Alkyl Benzoate | 10.0 | 0.0 | 0.0 | 0.0 | 0.0 | 0.0 | 0.0 | 0.0 | 0.0 |
| New UV filter | | 3.0 | 15.0 | 5.0 | 10.0 | 5.0 | 8.0 | 5.0 | 15.0 |
| Cyclotetrasiloxane (and) | 3.0 | 3.0 | 3.0 | 3.0 | 3.0 | 3.0 | 3.0 | 3.0 | 3.0 |
| Cyclopentasiloxane | | | | | | | | | |
| PEG/PPG-4/12 Dimethicone | 1.0 | 1.0 | 1.0 | 1.0 | 1.0 | 1.0 | 1.0 | 1.0 | 1.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| UV WAT | | 5.0 | 5.0 | 5.0 | 5.0 | 5.0 | 5.0 | 0.0 | 5.0 |

| | Gel Cream | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Sodium Carbomer | 0.50 | 0.50 | 0.50 | 0.50 | 0.50 | 0.50 | 0.50 | 0.50 | 0.50 |
| Acrylates/C10-C30 Alkyl | 1.0 | 1.0 | 1.0 | 1.0 | 1.0 | 1.0 | 1.0 | 1.0 | 1.0 |
| Acrylate | | | | | | | | | |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| UV WAT | | 5.0 | 5.0 | 5.0 | 5.0 | 5.0 | 5.0 | 0.0 | 5.0 |
| C12-15 Alkyl Benzoate | 2.0 | 0.0 | 0.0 | 0.0 | 0.0 | 0.0 | 0.0 | 0.0 | 0.0 |
| New UV filter | | 3.0 | 15.0 | 5.0 | 10.0 | 5.0 | 8.0 | 5.0 | 15.0 |
| Butylenglycol Dicaprylat/ | 4.0 | 0.0 | 0.0 | 0.0 | 0.0 | 0.0 | 0.0 | 0.0 | 0.0 |
| Dicaprate | | | | | | | | | |
| Cetyl Dimethicone | 2.0 | 2.0 | 2.0 | 2.0 | 2.0 | 2.0 | 2.0 | 2.0 | 2.0 |
| Glycerine | 0.50 | 0.50 | 0.50 | 0.50 | 0.50 | 0.50 | 0.50 | 0.50 | 0.50 |
| Tocopherol | 3.0 | 3.0 | 3.0 | 3.0 | 3.0 | 3.0 | 3.0 | 3.0 | 3.0 |
| Methylparabene | 1.0 | 1.0 | 1.0 | 1.0 | 1.0 | 1.0 | 1.0 | 1.0 | 1.0 |
| Phenoxyethanol | 1.0 | 1.0 | 1.0 | 1.0 | 1.0 | 1.0 | 1.0 | 1.0 | 1.0 |
| Water | ad | ad | ad | ad | ad | ad | ad | ad | ad |
| | 100 | 100 | 100 | 100 | 100 | 100 | 100 | 100 | 100 |

| | Emulsifier Free | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Acrylates/C10-30 Alkyl Acrylate | 0.40 | 0.40 | 0.40 | 0.40 | 0.40 | 0.40 | 0.40 | 0.40 | 0.40 |
| Crosspolymer | | | | | | | | | |
| Xanthan Gum | 0.10 | 0.10 | 0.10 | 0.10 | 0.10 | 0.10 | 0.10 | 0.10 | 0.10 |
| UV SOL | | 10.0 | 15.0 | 5.0 | 10.0 | 5.0 | 8.0 | 5.0 | 15.0 |
| UV LIQ | | 5.0 | 10.0 | 5.0 | 10.0 | 5.0 | 10.0 | 5.0 | 5.0 |
| UV WAT | | 5.0 | 5.0 | 10.0 | 10.0 | 15.0 | 15.0 | 15.0 | 0.0 |
| New UV filter | | 3.0 | 5.0 | 5.0 | 5.0 | 5.0 | 5.0 | 0.0 | 5.0 |
| C12-15 Alkyl Benzoate | 6.0 | 0.0 | 0.0 | 0.0 | 0.0 | 0.0 | 0.0 | 0.0 | 0.0 |
| Octyldodecanol | 7.0 | 0.0 | 0.0 | 0.0 | 0.0 | 0.0 | 0.0 | 0.0 | 0.0 |
| Cetyl Dimethicone | 6.0 | 6.0 | 6.0 | 6.0 | 6.0 | 6.0 | 6.0 | 6.0 | 6.0 |
| Ethylhexyloxyglycerine | 1.0 | 1.0 | 1.0 | 1.0 | 1.0 | 1.0 | 1.0 | 1.0 | 1.0 |
| Butylen Glycol | 10.0 | 10.0 | 10.0 | 10.0 | 10.0 | 10.0 | 10.0 | 10.0 | 10.0 |
| Glycinc Soja | 1.0 | 1.0 | 1.0 | 1.0 | 1.0 | 1.0 | 1.0 | 1.0 | 1.0 |
| Vitamin E Acetate | 0.25 | 0.25 | 0.25 | 0.25 | 0.25 | 0.25 | 0.25 | 0.25 | 0.25 |
| Trisodium EDTA | 0.10 | 0.10 | 0.10 | 0.10 | 0.10 | 0.10 | 0.10 | 0.10 | 0.10 |
| Ethanol | 3.50 | 3.50 | 3.50 | 3.50 | 3.50 | 3.50 | 3.50 | 3.50 | 3.50 |
| Parfume | 0.20 | 0.20 | 0.20 | 0.20 | 0.20 | 0.20 | 0.20 | 0.20 | 0.20 |
| Watersoluble Dyes | 0.02 | 0.02 | 0.02 | 0.02 | 0.02 | 0.02 | 0.02 | 0.02 | 0.02 |
| Water | ad | ad | ad | ad | ad | ad | ad | ad | ad |
| | 100 | 100 | 100 | 100 | 100 | 100 | 100 | 100 | 100 |

| | | Sun Spray | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Ethyl Trisiloxane | 2.50 | 2.50 | 2.50 | 2.50 | 2.50 | 2.50 | 2.50 | 2.50 | 2.50 |
| | Hydrogenated Coco- | 1.50 | 1.50 | 1.50 | 1.50 | 1.50 | 1.50 | 1.50 | 1.50 | 1.50 |
| | glycerides | | | | | | | | | |
| | C12-15 Alkyl Benzoate | 3.60 | 3.60 | 3.60 | 3.60 | 3.60 | 3.60 | 3.60 | 3.60 | 3.60 |
| | New UV filter | | 5.0 | 3.0 | 5.0 | 5.0 | 5.0 | 5.0 | 0.0 | 5.0 |
| | UV SOL | | 10.0 | 15.0 | 5.0 | 10.0 | 5.0 | 8.0 | 5.0 | 15.0 |
| | UV LIQ | | 5.0 | 10.0 | 5.0 | 10.0 | 5.0 | 10.0 | 5.0 | 5.0 |
| Part B | Water (and) Caprylic/Capric | 10.0 | 10.0 | 10.0 | 10.0 | 10.0 | 10.0 | 10.0 | 10.0 | 10.0 |
| | Triglyceride (and) Glycerin | | | | | | | | | |
| | (and) Ceteareth-25 (and) | | | | | | | | | |
| | Disodium Ethylene Di(Cocamide | | | | | | | | | |
| | PEG-15 Disulfate) (and) | | | | | | | | | |
| | Sodium Lauroyl Lactylate | | | | | | | | | |
| | (and) Behenyl Alcohol (and) | | | | | | | | | |
| | Glyceryl Stearate (and) | | | | | | | | | |
| | Glyceryl Stearate Citrate | | | | | | | | | |
| | (and) Xanthan Gum | | | | | | | | | |
| | PVP/Hexadecene Copolymer | 0.50 | 0.50 | 0.50 | 0.50 | 0.50 | 0.50 | 0.50 | 0.50 | 0.50 |
| | Aqua | Qs to | Qs to | Qs to | Qs to | Qs to | Qs to | Qs to | Qs to | Qs to |
| | | 10 | 10 | 10 | 10 | 10 | 10 | 10 | 10 | 10 |
| | Glycerin | 3.0 | 3.0 | 3.0 | 3.0 | 3.0 | 3.0 | 3.0 | 3.0 | 3.0 |
| | UV WAT | | 5.0 | 5.0 | 10.0 | 10.0 | 15.0 | 15.0 | 15.0 | 0.0 |
| Part C | Alcohol Denatured | 7.0 | 7.0 | 7.0 | 7.0 | 7.0 | 7.0 | 7.0 | 7.0 | 7.0 |
| Part D | Phenoxyethanol (and) | 0.70 | 0.70 | 0.70 | 0.70 | 0.70 | 0.70 | 0.70 | 0.70 | 0.70 |
| | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| | Tocopheryl Acetate | 0.60 | 0.60 | 0.60 | 0.60 | 0.60 | 0.60 | 0.60 | 0.60 | 0.60 |

| | | Sun spray foaming | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Behenyl Alcohol (and) | 0.50 | 0.50 | 0.50 | 0.50 | 0.50 | 0.50 | 0.50 | 0.50 | 0.50 |
| | Glyceryl Stearate (and) | | | | | | | | | |
| | Glyceryl Stearate Citrate (and) | | | | | | | | | |
| | Disodium Ethylene | | | | | | | | | |
| | Di(Cocamide PEG-15 | | | | | | | | | |
| | Disulfate) | | | | | | | | | |
| | Isotrideceth-12 | 0.50 | 0.50 | 0.50 | 0.50 | 0.50 | 0.50 | 0.50 | 0.50 | 0.50 |
| | Hydrogenated | 1.50 | 0.0 | 0.0 | 0.0 | 0.0 | 0.0 | 0.0 | 0.0 | 0.0 |
| | Cocoglycerides | | | | | | | | | |
| | C12-15 Alkyl Benzoate | 8.0 | 0.0 | 0.0 | 0.0 | 0.0 | 0.0 | 0.0 | 0.0 | 0.0 |
| | New UV filter | | 5.0 | 5.0 | 5.0 | 5.0 | 5.0 | 5.0 | 0.0 | 5.0 |
| | UV SOL | | 3.0 | 15.0 | 5.0 | 10.0 | 5.0 | 8.0 | 5.0 | 15.0 |
| | UV LIQ | | 5.0 | 10.0 | 5.0 | 10.0 | 5.0 | 10.0 | 5.0 | 5.0 |
| Part B | Aqua | 5.0 | 5.0 | 5.0 | 5.0 | 5.0 | 5.0 | 5.0 | 5.0 | 5.0 |
| | Glycerin | 4.0 | 4.0 | 4.0 | 4.0 | 4.0 | 4.0 | 4.0 | 4.0 | 4.0 |
| | Galactoarabinan | 0.25 | 0.25 | 0.25 | 0.25 | 0.25 | 0.25 | 0.25 | 0.25 | 0.25 |
| | UV WAT | | 5.0 | 5.0 | 10.0 | 10.0 | 15.0 | 15.0 | 15.0 | 0.0 |
| Part C | Disodium Ethylene | 1.0 | 1.0 | 1.0 | 1.0 | 1.0 | 1.0 | 1.0 | 1.0 | 1.0 |
| | Di(Cocamide PEG-15 | | | | | | | | | |
| | Disulfate) (and) Sodium | | | | | | | | | |
| | Lauroyl Lactylate | | | | | | | | | |
| | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| Part D | Phenoxyethanol (and) | 0.70 | 0.70 | 0.70 | 0.70 | 0.70 | 0.70 | 0.70 | 0.70 | 0.70 |
| | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| | Tocopheryl Acetate | 0.60 | 0.60 | 0.60 | 0.60 | 0.60 | 0.60 | 0.60 | 0.60 | 0.60 |

| | Active Naturals Continuous Spray | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Acrylates/Octylacrylamide | 3.0 | 3.01 | 3.02 | 3.03 | 3.04 | 3.05 | 3.06 | 3.07 | 3.08 |
| Copolymer | | | | | | | | | |
| Ascorbyl Palmitate | 0.30 | 0.31 | 0.32 | 0.33 | 0.34 | 0.35 | 0.36 | 0.37 | 0.38 |
| Diisopropyl Adipate | 3.0 | 0.0 | 0.0 | 0.0 | 0.0 | 0.0 | 0.0 | 0.0 | 0.0 |
| Glycerine soja seed extract | 3.0 | 0.0 | 0.0 | 0.0 | 0.0 | 0.0 | 0.0 | 0.0 | 0.0 |
| soybean | | | | | | | | | |
| Isodecyl Neopentanoate | 2.50 | 0.0 | 0.0 | 0.0 | 0.0 | 0.0 | 0.0 | 0.0 | 0.0 |
| New UV filter | | 3.0 | 15.0 | 5.0 | 10.0 | 5.0 | 8.0 | 5.0 | 15.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| UV WAT | | 5.0 | 5.0 | 5.0 | 5.0 | 5.0 | 5.0 | 0.0 | 5.0 |
| Lauryl Lactate | 1.50 | 1.50 | 1.50 | 1.50 | 1.50 | 1.50 | 1.50 | 1.50 | 1.50 |
| PPG-12/SMDI Copolymer | 1.0 | 1.0 | 1.0 | 1.0 | 1.0 | 1.0 | 1.0 | 1.0 | 1.0 |
| Retinyl Palmitate | 0.30 | 0.30 | 0.30 | 0.30 | 0.30 | 0.30 | 0.30 | 0.30 | 0.30 |
| SD Alcohol 40 | 80% | 80% | 80% | 80% | 80% | 80% | 80% | 80% | 80% |
| | V/V | V/V | V/V | V/V | V/V | V/V | V/V | V/V | V/V |
| Tocopherol Acetate | 1.0 | 1.0 | 1.0 | 1.0 | 1.0 | 1.0 | 1.0 | 1.0 | 1.0 |
| Parfume | q.s. | q.s. | q.s. | q.s. | q.s. | q.s. | q.s. | q.s. | q.s. |
| Aqua | ad 100 | ad 100 | ad 100 | ad 100 | ad 100 | ad 100 | ad 100 | ad 100 | ad 100 |
| Propellent | Qs | Qs | Qs | Qs | Qs | Qs | Qs | Qs | Qs |

| | | W/O Sunscreen Lotion | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | PEG-7 Hydrogenated | 3.0 | 3.0 | 3.0 | 3.0 | 3.0 | 3.0 | 3.0 | 3.0 | 3.0 |
| | Castor Oil | | | | | | | | | |
| | Polyglyceryl-3 Diisostearate | 4.0 | 4.0 | 4.0 | 4.0 | 4.0 | 4.0 | 4.0 | 4.0 | 4.0 |
| | Microcrystalline Wax | 1.0 | 1.0 | 1.0 | 1.0 | 1.0 | 1.0 | 1.0 | 1.0 | 1.0 |
| | Magnesium Stearate | 1.50 | 1.50 | 1.50 | 1.50 | 1.50 | 1.50 | 1.50 | 1.50 | 1.50 |
| | Propylparaben | 0.10 | 0.10 | 0.10 | 0.10 | 0.10 | 0.10 | 0.10 | 0.10 | 0.10 |
| | Mineral Oil | 15.0 | 5.0 | 0.0 | 10.0 | 0.0 | 5.0 | 0.0 | 5.0 | 10.0 |
| | New UV filter | | 3.0 | 15.0 | 5.0 | 10.0 | 5.0 | 5.0 | 5.0 | 15.0 |
| | Octyldodecanol | 8.0 | 0.0 | 0.0 | 0.0 | 0.0 | 0.0 | 0.0 | 0.0 | 0.0 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part B | Water | qs to | qs to | qs to | qs to | qs to | qs to | qs to | qs to | qs to |
| | | 100 | 100 | 100 | 100 | 100 | 100 | 100 | 100 | 100 |
| | UV WAT | | 5.0 | 5.0 | 5.0 | 5.0 | 5.0 | 5.0 | 0.0 | 5.0 |
| Part C | Water (and) Citric Acid | 0.05 | 0.05 | 0.05 | 0.05 | 0.05 | 0.05 | 0.05 | 0.05 | 0.05 |
| | Methylparaben | 0.15 | 0.15 | 0.15 | 0.15 | 0.15 | 0.15 | 0.15 | 0.15 | 0.15 |
| | Magnesium Sulfate | 0.50 | 0.50 | 0.50 | 0.50 | 0.50 | 0.50 | 0.50 | 0.50 | 0.50 |
| | Fragrance | qs | qs | qs | qs | qs | qs | qs | qs | qs |

| | | W/Si sun cream | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Cyclopentasiloxane (and) | 3.0 | 3.0 | 3.0 | 3.0 | 3.0 | 3.0 | 3.0 | 3.0 | 3.0 |
| | Dimethicone/Vinyl | | | | | | | | | |
| | Dimethicone Crosspolymer | | | | | | | | | |
| | PEG-10 Dimethicone | 0.30 | 0.30 | 0.30 | 0.30 | 0.30 | 0.30 | 0.30 | 0.30 | 0.30 |
| | Polyglyceryl-3 Polydimethyl- | 4.0 | 4.0 | 4.0 | 4.0 | 4.0 | 4.0 | 4.0 | 4.0 | 4.0 |
| | siloxyethyl Dimethicone | | | | | | | | | |
| | Dimethicone | 5.0 | 5.0 | 5.0 | 5.0 | 5.0 | 5.0 | 5.0 | 5.0 | 5.0 |
| | Cyclomethicone | 15.0 | 15.0 | 15.0 | 15.0 | 15.0 | 15.0 | 15.0 | 15.0 | 15.0 |
| | New UV filter | | 3.0 | 15.0 | 5.0 | 10.0 | 5.0 | 5.0 | 5.0 | 15.0 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part C | 1.3-Butylen Glycol | 8.0 | 8.0 | 8.0 | 8.0 | 8.0 | 8.0 | 8.0 | 8.0 | 8.0 |
| | Sodium Citrate | 0.20 | 0.20 | 0.20 | 0.20 | 0.20 | 0.20 | 0.20 | 0.20 | 0.20 |
| | Ethyl Alcohol | 5.0 | 5.0 | 5.0 | 5.0 | 5.0 | 5.0 | 5.0 | 5.0 | 5.0 |
| | Sodium Chloride | 0.50 | 0.50 | 0.50 | 0.50 | 0.50 | 0.50 | 0.50 | 0.50 | 0.50 |
| | UV WAT | | 5.0 | 5.0 | 5.0 | 5.0 | 5.0 | 5.0 | 0.0 | 5.0 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

| | Lipstick | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Caprylic/Capric Triglyceride | 12.00 | 0.0 | 0.0 | 0.0 | 0.0 | 0.0 | 0.0 | 0.0 | 0.0 |
| New UV filter | | 3.0 | 15.0 | 5.0 | 10.0 | 5.0 | 5.0 | 5.0 | 15.0 |
| Octyldodecanol | 7.0 | 7.0 | 7.0 | 7.0 | 7.0 | 7.0 | 7.0 | 7.0 | 7.0 |
| Pentaerythrityl | 10.0 | 5.0 | 0.0 | 5.0 | 0.0 | 0.0 | 0.0 | 0.0 | 5.0 |
| Tetraisostearate | | | | | | | | | |
| Polyglyceryl-3 | 2.50 | 2.50 | 2.50 | 2.50 | 2.50 | 2.50 | 2.50 | 2.50 | 2.50 |
| Diisostearate | | | | | | | | | |
| Bis-Diglyceryl | 9.0 | 9.0 | 9.0 | 9.0 | 9.0 | 9.0 | 9.0 | 9.0 | 9.0 |
| Polyacyladipate-2 | | | | | | | | | |
| Cetearyl Alcohol | 8.0 | 8.0 | 8.0 | 8.0 | 8.0 | 8.0 | 8.0 | 8.0 | 8.0 |
| Myristyl Myristate | 3.50 | 3.50 | 3.50 | 3.50 | 3.50 | 3.50 | 3.50 | 3.50 | 3.50 |
| Beeswax | 5.0 | 5.0 | 5.0 | 5.0 | 5.0 | 5.0 | 5.0 | 5.0 | 5.0 |
| Copernicia Cerifera | 1.50 | 1.50 | 1.50 | 1.50 | 1.50 | 1.50 | 1.50 | 1.50 | 1.50 |
| (Carnauba) Wax | | | | | | | | | |
| Cera Alba | 0.50 | 0.50 | 0.50 | 0.50 | 0.50 | 0.50 | 0.50 | 0.50 | 0.50 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| Tocopheryl Acetate | 0.50 | 0.50 | 0.50 | 0.50 | 0.50 | 0.50 | 0.50 | 0.50 | 0.50 |
| Tocopherol; Ascorbyl | 0.05 | 0.05 | 0.05 | 0.05 | 0.05 | 0.05 | 0.05 | 0.05 | 0.05 |
| Palmitate | | | | | | | | | |
| Simmondsia Chinensis | 2.0 | 2.0 | 2.0 | 2.0 | 2.0 | 2.0 | 2.0 | 2.0 | 2.0 |
| (Jojoba) Seed Extract | | | | | | | | | |
| Parfum. BHT | q.s | q.s | q.s | q.s | q.s | q.s | q.s | q.s | q.s |
| Ricinus Communis | QS to | QS to | QS to | QS to | QS to | QS to | QS to | QS to | QS to |
| | 100 | 100 | 100 | 100 | 100 | 100 | 100 | 100 | 100 |

| | Waterproof Gel | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Anhydrous Ethanol | Qsp | Qsp | Qsp | Qsp | Qsp | Qsp | Qsp | Qsp | Qsp |
| | 100 | 100 | 100 | 100 | 100 | 100 | 100 | 100 | 100 |
| Hydroxypropyl Cellulose | 1.0 | 1.0 | 1.0 | 1.0 | 1.0 | 1.0 | 1.0 | 1.0 | 1.0 |
| Acrylates/Octylacrylamide | 1.0 | 1.0 | 1.0 | 1.0 | 1.0 | 1.0 | 1.0 | 1.0 | 1.0 |
| Copolymer | | | | | | | | | |
| New UV filter | | 3.0 | 15.0 | 5.0 | 10.0 | 5.0 | 5.0 | 5.0 | 15.0 |
| C12-15 Alkyl Benzoate | 10.0 | 10.0 | 10.0 | 10.0 | 10.0 | 10.0 | 10.0 | 10.0 | 10.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| Cyclomethicone | 3.0 | 3.0 | 3.0 | 3.0 | 3.0 | 3.0 | 3.0 | 3.0 | 3.0 |
| PEG/PPG-4/12 Dimethicone | 1.0 | 1.0 | 1.0 | 1.0 | 1.0 | 1.0 | 1.0 | 1.0 | 1.0 |

| | SUNSCREEN Oleogel | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Isopropyl Myristate | 38.0 | qs. | qs. | qs. | qs. | qs. | qs. | qs. | qs. |
| | | 100 | 100 | 100 | 100 | 100 | 100 | 100 | 100 |
| C12-15 Alkyl Benzoate | 10.0 | 5.0 | 5.0 | 5.0 | 5.0 | 5.0 | 5.0 | 5.0 | 5.0 |
| New UV filter | | 3.0 | 15.0 | 5.0 | 10.0 | 5.0 | 5.0 | 5.0 | 15.0 |
| Caprylic/Capric Triglyceride | 39.0 | 15.0 | 15.0 | 15.0 | 15.0 | 15.0 | 15.0 | 15.0 | 15.0 |
| Perfume | q.s. | q.s. | q.s. | q.s. | q.s. | q.s. | q.s. | q.s. | q.s. |
| Disteardimonium Hectorite | 15.0 | 15.0 | 15.0 | 15.0 | 15.0 | 15.0 | 15.0 | 15.0 | 15.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| Propylene Carbonate | 5.0 | 5.0 | 5.0 | 5.0 | 5.0 | 5.0 | 5.0 | 5.0 | 5.0 |

| | | O/W/O Soft Cream | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | PEG-60 Hydrogenated | 0.40 | 0.40 | 0.40 | 0.40 | 0.40 | 0.40 | 0.40 | 0.40 | 0.40 |
| | Castor Oil | | | | | | | | | |
| | Water | 0.90 | 0.90 | 0.90 | 0.90 | 0.90 | 0.90 | 0.90 | 0.90 | 0.90 |
| Part B | Tocopheryl Acetate | 2.0 | 2.0 | 2.0 | 2.0 | 2.0 | 2.0 | 2.0 | 2.0 | 2.0 |
| | Retinyl Palmitate | 0.50 | 0.50 | 0.50 | 0.50 | 0.50 | 0.50 | 0.50 | 0.50 | 0.50 |
| | Caprylic/Capric Triglyceride | 2.50 | 2.50 | 2.50 | 2.50 | 2.50 | 2.50 | 2.50 | 2.50 | 2.50 |
| Part C | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Sodium Chloride | 0.40 | 0.40 | 0.40 | 0.40 | 0.40 | 0.40 | 0.40 | 0.40 | 0.40 |
| | UV WAT | | 5.0 | 5.0 | 5.0 | 5.0 | 5.0 | 5.0 | 0.0 | 5.0 |
| Part D | Cetyl PEG/PPG-10/1 | 2.0 | 2.0 | 2.0 | 2.0 | 2.0 | 2.0 | 2.0 | 2.0 | 2.0 |
| | Dimethicone | | | | | | | | | |
| | Microcrystalline Wax | 0.90 | 0.90 | 0.90 | 0.90 | 0.90 | 0.90 | 0.90 | 0.90 | 0.90 |
| | Hydrogenated Castor Oil | 0.60 | 0.60 | 0.60 | 0.60 | 0.60 | 0.60 | 0.60 | 0.60 | 0.60 |
| | Decyl Oleate | 10.5 | 5.0 | 5.0 | 5.0 | 5.0 | 5.0 | 5.0 | 5.0 | 5.0 |
| | Caprylic/Capric Triglyceride | 10.0 | 0.0 | 0.0 | 0.0 | 0.0 | 0.0 | 0.0 | 0.0 | 0.0 |
| | Jojoba (Buxus Chinensis) Oil | 2.0 | 2.0 | 2.0 | 2.0 | 2.0 | 2.0 | 2.0 | 2.0 | 2.0 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| | New UV filter | | 3.0 | 15.0 | 5.0 | 10.0 | 5.0 | 5.0 | 5.0 | 15.0 |
| | Preservative, Parfum | q.s. | q.s. | q.s. | q.s. | q.s. | q.s. | q.s. | q.s. | q.s. |

| | W/O/W Emulsion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Glycerylstearate | 3.0 | 3.0 | 3.0 | 3.0 | 3.0 | 3.0 | 3.0 | 3.0 | 3.0 |
| PEG-100-Stearate | 0.75 | 0.75 | 0.75 | 0.75 | 0.75 | 0.75 | 0.75 | 0.75 | 0.75 |
| Behenylalcohol | 2.0 | 2.0 | 2.0 | 2.0 | 2.0 | 2.0 | 2.0 | 2.0 | 2.0 |
| Caprylic-/Capric- | 8.0 | 0.0 | 0.0 | 0.0 | 0.0 | 0.0 | 0.0 | 0.0 | 0.0 |
| Triglyceride | | | | | | | | | |
| Octyldodecanol | 5.0 | 0.0 | 0.0 | 0.0 | 0.0 | 0.0 | 0.0 | 0.0 | 0.0 |
| C12-15 Alkylbenzoate | 3.0 | 0.0 | 0.0 | 0.0 | 0.0 | 0.0 | 0.0 | 0.0 | 0.0 |
| New UV filter | | 3.0 | 15.0 | 5.0 | 10.0 | 5.0 | 5.0 | 5.0 | 15.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| UV WAT | | 5.0 | 5.0 | 5.0 | 5.0 | 5.0 | 5.0 | 0.0 | 5.0 |
| Dodecanedioic Acid | 1.0 | 1.0 | 1.0 | 1.0 | 1.0 | 1.0 | 1.0 | 1.0 | 1.0 |
| Magnesium Sulfate | 0.80 | 0.80 | 0.80 | 0.80 | 0.80 | 0.80 | 0.80 | 0.80 | 0.80 |
| EDTA | 0.10 | 0.10 | 0.10 | 0.10 | 0.10 | 0.10 | 0.10 | 0.10 | 0.10 |
| Phenoxyethanol | q.s. | q.s. | q.s. | q.s. | q.s. | q.s. | q.s. | q.s. | q.s. |
| Parfume | q.s. | q.s. | q.s. | q.s. | q.s. | q.s. | q.s. | q.s. | q.s. |
| Water | ad 100 | ad 10.0 | ad 100 | ad 100 | ad 100 | ad 100 | ad 100 | ad 100 | ad 100 |
| PH-value adjusted to 6.0 | | | | | | | | | |

| | Cream-to-powder | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Isoeicosane | 10.0 | 5.0 | 5.0 | 5.0 | 5.0 | 5.0 | 5.0 | 5.0 | 5.0 |
| Polyisobutene | 2.0 | 2.0 | 2.0 | 2.0 | 2.0 | 2.0 | 2.0 | 2.0 | 2.0 |
| New UV filter | | 3.0 | 15.0 | 5.0 | 10.0 | 5.0 | 5.0 | 5.0 | 15.0 |
| Cetearyl Octanoate | 20.5 | 10.0 | 5.0 | 10.0 | 5.0 | 5.0 | 5.0 | 5.0 | 10.0 |
| Oleyl alcohol | 5.0 | 5.0 | 5.0 | 5.0 | 5.0 | 5.0 | 5.0 | 5.0 | 5.0 |
| Ceresin | 4.50 | 4.50 | 4.50 | 4.50 | 4.50 | 4.50 | 4.50 | 4.50 | 4.50 |
| Talc | 11.60 | 11.60 | 11.60 | 11.60 | 11.60 | 11.60 | 11.60 | 11.60 | 11.60 |
| Polyethylene | 4.0 | 4.0 | 4.0 | 4.0 | 4.0 | 4.0 | 4.0 | 4.0 | 4.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| Silica | 17.75 | 17.75 | 17.75 | 17.75 | 17.75 | 17.75 | 17.75 | 17.75 | 17.75 |
| Calcium Aluminum Boro- | 1.0 | 1.0 | 1.0 | 1.0 | 1.0 | 1.0 | 1.0 | 1.0 | 1.0 |
| silicate (and) Bismuth | | | | | | | | | |
| oxychloride | | | | | | | | | |
| Iron Oxides | 3.40 | 3.40 | 3.40 | 3.40 | 3.40 | 3.40 | 3.40 | 3.40 | 3.40 |
| Tocopherol Acetate | 0.25 | 0.25 | 0.25 | 0.25 | 0.25 | 0.25 | 0.25 | 0.25 | 0.25 |

| | Foundations: Anhydrous forms | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Isononyl Isononanoate | qsp | qsp | qsp | qsp | qsp | qsp | qsp | qsp | qsp |
| New UV filter | 3.0 | 3.0 | 15.0 | 5.0 | 10.0 | 5.0 | 5.0 | 5.0 | 15.0 |
| Sorbitan Sesquioleate | 0.50 | 0.50 | 0.50 | 0.50 | 0.50 | 0.50 | 0.50 | 0.50 | 0.50 |
| Cyclopentasiloxane | 15.0 | 15.0 | 15.0 | 15.0 | 15.0 | 15.0 | 15.0 | 15.0 | 15.0 |
| Cylopentasiloxane (and) | 15.0 | 15.0 | 15.0 | 15.0 | 15.0 | 15.0 | 15.0 | 15.0 | 15.0 |
| Quaternium-18 Hectorite | | | | | | | | | |
| Talc | 5.0 | 5.0 | 5.0 | 5.0 | 5.0 | 5.0 | 5.0 | 5.0 | 5.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| Iron oxides | 2.50 | 2.50 | 2.50 | 2.50 | 2.50 | 2.50 | 2.50 | 2.50 | 2.50 |
| PVP/eicosane copolymer | 1.0 | 1.0 | 1.0 | 1.0 | 1.0 | 1.0 | 1.0 | 1.0 | 1.0 |
| Tocopherol Acetate | 0.50 | 0.50 | 0.50 | 0.50 | 0.50 | 0.50 | 0.50 | 0.50 | 0.50 |

| | Pickering Emulsions | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| Octyidodecanol | 5.50 | 0.0 | | | | | | | |
| New UV filter | 3.0 | 3.0 | 15.0 | 5.0 | 10.0 | 5.0 | 5.0 | 5.0 | 15.0 |
| C12-15-Alkyl Benzoate | 6.50 | 0.0 | 0.0 | 0.0 | 0.0 | 0.0 | 0.0 | 0.0 | 0.0 |
| Dicaprylyl Ether | 5.50 | 0.0 | 0.0 | 0.0 | 0.0 | 0.0 | 0.0 | 0.0 | 0.0 |
| Hydroxyoctacosanyl | 2.0 | 2.0 | 2.0 | 2.0 | 2.0 | 2.0 | 2.0 | 2.0 | 2.0 |
| Hydroxystearate | | | | | | | | | |
| Disteardimonium Hectorit | 1.0 | 1.0 | 1.0 | 1.0 | 1.0 | 1.0 | 1.0 | 1.0 | 1.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| UV WAT | | 5.0 | 5.0 | 5.0 | 5.0 | 5.0 | 5.0 | 0.0 | 5.0 |
| Bariumsulfate | 0.50 | 0.50 | 0.50 | 0.50 | 0.50 | 0.50 | 0.50 | 0.50 | 0.50 |
| Boron Nitride | 2.0 | 2.0 | 2.0 | 2.0 | 2.0 | 2.0 | 2.0 | 2.0 | 2.0 |
| NaCl | 1.0 | 1.0 | 1.0 | 1.0 | 1.0 | 1.0 | 1.0 | 1.0 | 1.0 |
| Glycerine | 10.0 | 10.0 | 10.0 | 10.0 | 10.0 | 10.0 | 10.0 | 10.0 | 10.0 |
| Trisodlum EDTA | 1.0 | 1.0 | 1.0 | 1.0 | 1.0 | 1.0 | 1.0 | 1.0 | 1.0 |
| Porpylene Carbonate | 0.33 | 0.33 | 0.33 | 0.33 | 0.33 | 0.33 | 0.33 | 0.33 | 0.33 |
| Methylparabene | 0.20 | 0.20 | 0.20 | 0.20 | 0.20 | 0.20 | 0.20 | 0.20 | 0.20 |
| Phenoxyethanol | 0.50 | 0.50 | 0.50 | 0.50 | 0.50 | 0.50 | 0.50 | 0.50 | 0.50 |
| Hexamidine Diisethionate | 0.08 | 0.08 | 0.08 | 0.08 | 0.08 | 0.08 | 0.08 | 0.08 | 0.08 |
| Parfume | 0.40 | 0.40 | 0.40 | 0.40 | 0.40 | 0.40 | 0.40 | 0.40 | 0.40 |
| Water | ad | ad | ad | ad | ad | ad | ad | ad | ad |
| | 100 | 100 | 100 | 100 | 100 | 100 | 100 | 100 | 100 |

| | Microemulsion Lotion | | | | | | | | |
|---|---|---|---|---|---|---|---|---|---|
| | A | B | C | D | E | F | G | H | I |
| | % | % | % | % | % | % | % | % | % |
| INCI-Name | w/w | w/w | w/w | w/w | w/w | w/w | w/w | w/w | w/w |
| PPG-26-Buteth-26 | 3.0 | 3.0 | 3.0 | 3.0 | 3.0 | 3.0 | 3.0 | 3.0 | 3.0 |
| Ceteareth 20 | 10.0 | 10.0 | 10.0 | 10.0 | 10.0 | 10.0 | 10.0 | 10.0 | 10.0 |
| C12-15 Alkyl Benzoate | 5.0 | 0.0 | 0.0 | 0.0 | 0.0 | 0.0 | 0.0 | 0.0 | 0.0 |
| New UV filter | 3.0 | 3.0 | 15.0 | 5.0 | 10.0 | 5.0 | 5.0 | 5.0 | 15.0 |
| Oleth-5 | 4.0 | 4.0 | 4.0 | 4.0 | 4.0 | 4.0 | 4.0 | 4.0 | 4.0 |
| UV SOL | | 5.0 | 10.0 | 5.0 | 10.0 | 5.0 | 10.0 | 5.0 | 5.0 |
| UV LIQ | | 5.0 | 5.0 | 10.0 | 10.0 | 15.0 | 15.0 | 15.0 | 0.0 |
| PPG-11 Stearyl Ether | 10.0 | 0.0 | 0.0 | 0.0 | 0.0 | 0.0 | 0.0 | 0.0 | 0.0 |
| UV WAT | | 5.0 | 5.0 | 5.0 | 5.0 | 5.0 | 5.0 | 0.0 | 5.0 |
| Aluminum Chlorohydrex PG | 15.0 | 15.0 | 15.0 | 15.0 | 15.0 | 15.0 | 15.0 | 15.0 | 15.0 |
| Water | Qs to | Qs to | Qs to | Qs to | Qs to | Qs to | Qs to | Qs to | Qs to |
| | 100 | 100 | 100 | 100 | 100 | 100 | 100 | 100 | 100 |

| | | Cationic O/W sun cream | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Palmitamidopropyl- | 6.0 | 6.0 | 6.0 | 6.0 | 6.0 | 6.0 | 6.0 | 6.0 | 6.0 |
| | trimonium Chloride | | | | | | | | | |
| | Stearyl Alcohol | 2.0 | 2.0 | 2.0 | 2.0 | 2.0 | 2.0 | 2.0 | 2.0 | 2.0 |
| | Isocetyl Palmitate | 4.0 | 0.0 | 0.0 | 0.0 | 0.0 | 0.0 | 0.0 | 0.0 | 0.0 |
| | Decyl Cocoate | 3.0 | 0.0 | 0.0 | 0.0 | 0.0 | 0.0 | 0.0 | 0.0 | 0.0 |
| | C12-15 Alkyl Benzoate | 3.0 | 0.0 | 0.0 | 0.0 | 0.0 | 0.0 | 0.0 | 0.0 | 0.0 |
| | New UV filter | 3.0 | 3.0 | 15.0 | 5.0 | 10.0 | 5.0 | 5.0 | 5.0 | 15.0 |
| | Cetyl Dimethicone | 0.75 | 0.75 | 0.75 | 0.75 | 0.75 | 0.75 | 0.75 | 0.75 | 0.75 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part B | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Glycerin | 3.0 | 3.0 | 3.0 | 3.0 | 3.0 | 3.0 | 3.0 | 3.0 | 3.0 |
| | Trisodium EDTA | 0.10 | 0.10 | 0.10 | 0.10 | 0.10 | 0.10 | 0.10 | 0.10 | 0.10 |
| | UV WAT | | 5.0 | 5.0 | 5.0 | 5.0 | 5.0 | 5.0 | 0.0 | 5.0 |
| Part C | Capryl/Capramidopropyl | 8.0 | 8.0 | 8.0 | 8.0 | 8.0 | 8.0 | 8.0 | 8.0 | 8.0 |
| | Betaine | | | | | | | | | |
| | Phenoxyethanol (and) | 0.70 | 0.70 | 0.70 | 0.70 | 0.70 | 0.70 | 0.70 | 0.70 | 0.70 |
| | Methylparaben (and) | | | | | | | | | |
| | Ethylparaben (and) | | | | | | | | | |
| | Butylparaben (and) | | | | | | | | | |
| | Propylparaben (and) | | | | | | | | | |
| | Isobutylparaben | | | | | | | | | |
| | Tocopheryl Acetate | 0.80 | 0.80 | 0.80 | 0.80 | 0.80 | 0.80 | 0.80 | 0.80 | 0.80 |

| | | Si/W sun cream | | | | | | | | |
|---|---|---|---|---|---|---|---|---|---|---|
| | | A | B | C | D | E | F | G | H | I |
| | | % | % | % | % | % | % | % | % | % |
| | INCI-Name | w/w | w/w | w/w | w/w | w/w | w/w | w/w | w/w | w/w |
| Part A | Cyclopentasiloxane (and) | 2.0 | 2.0 | 2.0 | 2.0 | 2.0 | 2.0 | 2.0 | 2.0 | 2.0 |
| | Dimethicone/Vinyl | | | | | | | | | |
| | Dimethicone Crosspolymer | | | | | | | | | |
| | Dimethicone (and) Di- | 3.0 | 3.0 | 3.0 | 3.0 | 3.0 | 3.0 | 3.0 | 3.0 | 3.0 |
| | methicone/Vinyl Dimethicone | | | | | | | | | |
| | Crosspolymer | | | | | | | | | |
| | Cyclopentasiloxane | 10.0 | 10.0 | 10.0 | 10.0 | 10.0 | 10.0 | 10.0 | 10.0 | 10.0 |
| | New UV filter | 3.0 | 3.0 | 15.0 | 5.0 | 10.0 | 5.0 | 5.0 | 5.0 | 15.0 |
| | UV SOL | | 5.0 | 10.0 | 5.0 | 10.0 | 5.0 | 10.0 | 5.0 | 5.0 |
| | UV LIQ | | 5.0 | 5.0 | 10.0 | 10.0 | 15.0 | 15.0 | 15.0 | 0.0 |
| Part B | 1.3-Butylen Glycol | 3.0 | 3.0 | 3.0 | 3.0 | 3.0 | 3.0 | 3.0 | 3.0 | 3.0 |
| | Polyglyceryl-3 Disiloxane | 0.60 | 0.60 | 0.60 | 0.60 | 0.60 | 0.60 | 0.60 | 0.60 | 0.60 |
| | Dimethicone | | | | | | | | | |
| | Polyglyceryl-3 Polydi- | 0.30 | 0.30 | 0.30 | 0.30 | 0.30 | 0.30 | 0.30 | 0.30 | 0.30 |
| | methylsiloxyethyl | | | | | | | | | |
| | Dimethicone | | | | | | | | | |
| | Acrylamide/Sodium Acryloyl- | 0.60 | 0.60 | 0.60 | 0.60 | 0.60 | 0.60 | 0.60 | 0.60 | 0.60 |
| | dimethyltaurate Copolymer | | | | | | | | | |
| | (and) Isohexadecane | | | | | | | | | |
| | (and) Polysorbate 80 | | | | | | | | | |
| | Ammonium Acryloyldi- | 0.65 | 0.65 | 0.65 | 0.65 | 0.65 | 0.65 | 0.65 | 0.65 | 0.65 |
| | methyltaurate/VP Copolymer | | | | | | | | | |
| | Sodium Chloride | 0.10 | 0.10 | 0.10 | 0.10 | 0.10 | 0.10 | 0.10 | 0.10 | 0.10 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | UV WAT | | 5.0 | 5.0 | 5.0 | 5.0 | 5.0 | 5.0 | 0.0 | 5.0 |

The invention relates also to cosmetic compositions that comprise at least one of the UV absorbers according to the invention. The cosmetic compositions are suitable especially as UV filters, that is to say for the protection of organic materials that are sensitive to ultraviolet light, especially skin and hair, against the damaging action of UV radiation.

The UV absorbers can be used either in the dissolved state or in the micronized state.

The cosmetic compositions contain, for example, from 0.1 to 30% by weight, preferably from 0.1 to 15% by weight and especially from 0.5 to 10% by weight, based on the total weight of the composition, of one or more UV absorbers and at least one cosmetically tolerable adjuvant.

The cosmetic compositions can be prepared by physically mixing the UV absorber(s) with the adjuvant using customary methods, for example by simply stirring together the individual components, especially by making use of the dissolution properties of already known cosmetic UV absorbers, for example OMC, salicylic acid isooctyl ester, inter alia. The UV absorber can be used, for example, without further treatment, or in the micronized state, or in the form of a powder.

The cosmetic compositions may be, for example, creams, gels, lotions, alcoholic and aqueous/alcoholic solutions, emulsions, wax/fat compositions, stick preparations, powders or ointments.

The compositions according to the invention, for example creams, gels, lotions, alcoholic and aqueous/alcoholic solutions, emulsions, wax/fat compositions, stick preparations, powders or ointments, may in addition contain, as further adjuvants and additives, mild surfactants, super-fatting agents, pearlescent waxes, consistency regulators, thickeners, polymers, silicone compounds, fats, waxes, stabilisers, biogenic active ingredients, deodorising active ingredients, anti-dandruff agents, film formers, swelling agents, further UV light-protective factors, antioxidants, hydrotropic agents, preservatives, insect repellents, self-tanning agents, solubilisers, perfume oils, colourants, bacteria-inhibiting agents and the like.

Cosmetic formulations according to the invention are contained in a wide variety of cosmetic preparations. There come into consideration, for example, especially the following preparations: skin-care preparations, bath preparations, skin-care preparations, cosmetic personal care preparations, foot-care preparations, light-protective preparations, skin-tanning preparations, depigmenting preparations, insect-repellents, deodorants, antiperspirants, preparations for cleansing and caring for blemished skin, hair-removal preparations in chemical form (depilation), shaving preparations, fragrance preparations, cosmetic hair-treatment preparations.

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellent gas or pump-action spray) or an aerosol,
in the form of a foam, or
in the form of a paste.

Of special importance as cosmetic compositions for the skin are light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, sun protection oils, sun protection milk and sun protection preparations in the form of a spray.

Of special importance as cosmetic compositions for the hair are the above-mentioned preparations for hair treatment, especially hair-washing preparations in the form of shampoos, hair conditioners, hair-care preparations, e.g. pretreatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

The cosmetic preparation according to the invention contains from 0.1 to 15% by weight, preferably from 0.5 to 10% by weight, based on the total weight of the composition, of a UV absorber of formula (1) or of a mixture of UV absorbers and a cosmetically tolerable adjuvant.

The cosmetic preparation can be prepared by physically mixing the UV absorber or UV absorbers with the adjuvant using conventional methods, for example by simply stirring the individual components together.

The cosmetic preparation according to the invention can be formulated as a water-in-oil or oil-in-water emulsion, as an oil-in-alcohol lotion, as a vesicular dispersion of an ionic or non-ionic amphiphilic lipid, as a gel, solid stick or as an aerosol formulation.

As a water-in-oil or oil-in-water emulsion, the cosmetically tolerable adjuvant preferably contains from 5 to 50% of an oil phase, from 5 to 20% of an emulsifier and from 30 to 90% water. The oil phase can comprise any oil suitable for cosmetic formulations, for example one or more hydrocarbon oils, a wax, a natural oil, a silicone oil, a fatty acid ester or a fatty alcohol. Preferred mono- or poly-ols are ethanol, isopropanol, propylene glycol, hexylene glycol, glycerol and sorbitol.

For the cosmetic preparation according to the invention it is possible to use any conventionally usable emulsifier, for example one or more ethoxylated esters of natural derivatives, for example polyethoxylated esters of hydrogenated castor oil, or a silicone oil emulsifier, for example silicone polyol; an unethoxylated or ethoxylated fatty acid soap; an ethoxylated fatty alcohol; an unethoxylated or ethoxylated sorbitan ester; an ethoxylated fatty acid; or an ethoxylated glyceride.

The cosmetic preparation according to the invention is distinguished by excellent protection of human skin against the damaging effect of sunlight.

The benzylidene malonates according to the present invention may also be encapsulated according to methods known to a person that is skilled in the art, as described for example in WO2007/093252 or WO2009/012871 or WO2009/007264. The encapsulated benzylidene malonates thus obtained may also be used in the same way as the parent benzylidene malonates according to the present invention.

The benzylidene malonates according to the present invention may also be used to stabilize common ingredients of cosmetic formulations as described above. For example, the benzylidene malonates according to the present invention may be used to stabilize the UV-A filter Butyl Methoxydibenzoylmethane (Parsol 1789, CAS No. 70356-09-1).

The dimeric benzylidene malonates according to formula (1) are suitable especially as UV filters for the protection of polymeric materials. The UV filters are preferably used in polymeric substrates as disclosed in WO 2006/058856 on p. 4, I. 20 to p. 10, I. 15.

The polymeric substrates of the present invention comprise for example:

polymers of monoolefins and diolefins, and mixtures thereof, copolymers of monoolefins and diolefins with each other or with other vinyl monomers, hydrocarbon resins, polystyrene, aromatic homopolymers and copolymers derived from vinyl aromatic monomers, hydrogenated aromatic polymers derived from hydrogenation of polymers, hydrogenated aromatic polymers derived from hydrogenation of polymers mentioned, graft copolymers of vinyl aromatic monomers, halogen-containing polymers such as, polymers derived from unsaturated acids and derivatives thereof, polymers derived from unsaturated alcohols and amines or the acyl derivatives or acetals thereof, homopolymers and copolymers of cyclic ethers, polyacetals, polyphenylene oxides and sulfides, and mixtures of polyphenylene oxides with styrene polymers or polyamides, polyurethanes, polyamides and copolyamides, polyureas, polyimides, polyamide-imides, polyetherimids, polyesterimids, polyhydantoins and polybenzimidazoles, polyesters, polycarbonates and polyester carbonates, polyketones, polysulfones, polyether sulfones and polyether ketones, crosslinked polymers derived from aldehydes on the one hand and phenols, ureas and melamines on the other hand, drying and non-drying alkyd resins, unsaturated polyester resins derived from copolyesters of saturated and unsaturated dicarboxylic acids with polyhydric alcohols and vinyl compounds as crosslinking agents, and also halogen-containing modifications thereof of low flammability, crosslinkable acrylic resins derived from substituted acrylates, for example epoxy acrylates, urethane acrylates or polyester acrylates, alkyd resins, polyester resins and acrylate resins crosslinked with melamine resins, urea resins, isocyanates, isocyanurates, polyisocyanates or epoxy resins, crosslinked epoxy resins derived from aliphatic, cycloaliphatic, heterocyclic or aromatic glycidyl compounds or natural polymers.

The present polymeric substrates are used for example in moldings, rotomolded articles, injection molded articles, blow molded articles, films, tapes, mono-filaments, fibers, nonwovens, profiles, adhesives or putties, surface coatings and the like.

For example, the present PVC applications are employed for construction articles such as roofing and siding. The present polymeric substrates are used in polymer films in automotive windshields, other automotive glass, and in home and office windows.

The benzylidene malonates according to the present invention are also useful for stabilising body-care and household products, in particular used for skin-care products, bath and shower additives, preparations containing fragrances and odoriferous substances, hair-care products, dentifrices, deodorising and antiperspirant preparations, decorative preparations, light protection formulations and preparations containing active ingredients.

The body-care products can be in the form of creams, ointments, pastes, foams, gels, lotions, powders, make-ups, sprays, sticks or aerosols. They preferably contain the benzylidene malonates according to the present invention and, optionally, in admixture with other light stabilisers in the oil phase or in the aqueous or aqueous/alcoholic phase.

The following Table lists typical examples of body-care products of this invention and their ingredients:

| Body-care product | Ingredients |
|---|---|
| moisturising cream | vegetable oil, emulsifier, thickener, perfume, water, |
| | UV absorber |
| shampoo | surfactant, emulsifier, preservatives, perfume, |
| | UV absorber |
| toothpaste | cleaning agent, thickener, sweetener, flavour, |
| | colourant, UV absorber |
| lip-care stick | vegetable oil, wax, TiO₂, UV absorber |

Typical examples of novel household cleaning and treating agents are:

| Household cleaners/ | |
|---|---|
| household treating agents | Ingredients |
| detergent concentrate | surfactant mixture, ethanol, UV absorber, |
| | water |
| shoe polish | wax, wax emulsifier, UV absorber, water, |
| | preservative |
| wax-containing floor | emulsifier, wax, sodium chloride, UV absorber, |
| cleaning agent | water, preservative |

The benzylidene malonates are usually incorporated by dissolution in an oil phase or alcoholic or water phase, where required at elevated temperature

In the following Examples percentages relate to weight. The amounts of the benzylidene malonates compounds used relate to the pure substance.

### PREPARATION EXAMPLES OF THE NOVEL COMPOUNDS

### Example 1

### Preparation of the Compound of Formula

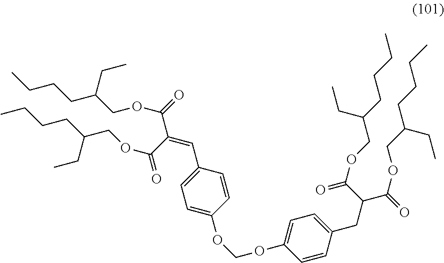

Di-(2-ethylhexyl)-malonate is obtained by transesterification of diethyl malonate with 2-ethyl hexanol by addition of H₂SO₄ (conc.) as described in US 2007/059258.

15.0 g of di-(2-ethylhexyl)malonate, 6.63 g of 4-hydroxybenzaldehyde, 0.45 ml of piperidine and 0.28 ml of acetic acid are heated for 4 h under reflux.

After cooling down the organic phase is washed with water and the solvent is removed in vacuum.

The residue is purified by column chromatography on silica gel (eluent cyclohexane/ethyl acetate 3:1).

13.0 g of (4-hydroxybenzylidene)-malonic acid-di-(2-ethylhexyl)-ester of formula

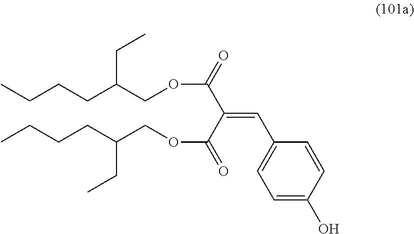

are obtained.

¹H-NMR (360 MHz, CDCl₃) δ=0.86 (t, J=7.0 Hz, 6H), 0.90 (t, J=7.0 Hz, 6H), 1.19-1.41 (m, 16H), 1.55-1.68 (m, 2H), 4.10-4.22 (m, 4H), 6.08 (bs, 1H), 6.79 (d, J=8.0 Hz, 2H), 7.32 (d, J=8.0 Hz, 2H), 7.64 (s, 1H).

2.35 g of potassium carbonate is added to a solution of 6.05 g of (4-hydroxybenzylidene)-malonic acid-di-(2-ethylhexyl)-ester (compound (101a)) and 1.85 g of crown ether [18]-crown-6 in 150 ml CH₂Cl₂ and the obtained suspension is heated for 20 h under reflux. The solvent is removed in vacuum, the residue dissolved in tert-butyl-methyl ether and washed successively with water and saturated aqueous NaCl solution.

The organic phase is dried with Na₂SO₄ and the solvent is removed in the vacuum.

5.3 g of 2-(4-(4-(2,2-bis-ethoxycarbonyl-vinyl)-phenoxymethoxy)-benzylidene)-malonic acid-di-(2-ethylhexyl)-ester according to formula (101) are obtained.

¹H-NMR (360 MHz, CDCl₃) δ=0.84 (t, J=7.0 Hz, 12H), 0.90 (t, J=7.0 Hz, 12H), 1.15-1.42 (m, 32H), 1.52-1.68 (m, 4H), 4.10-4.20 (m, 8H), 5.75 (s, 2H), 7.07 (d, J=8.5 Hz, 4H), 7.41 (d, J=8.5 Hz, 4H), 7.65 (s, 2H).

### Example 2

### Preparation of the Compound of Formula

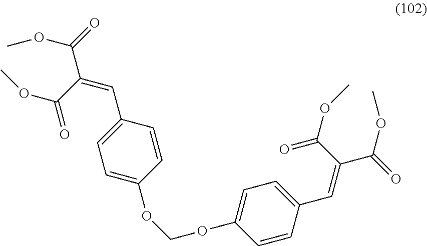

Analogous to example 1 the compound of the formula

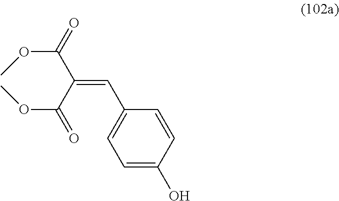

is obtained by Knoevenagel condensation of dimethylmalonate (59.7 g) with 4-hydroxybenzaldehyde (55.8 g) in a yield of 55.7 g.

¹H-NMR (360 MHz, d₆-acetone) δ=3.78 (s, 3H), 3.83 (s, 3H), 6.91 (d, J=8.5 Hz, 2H), 7.41 (d, J=8.5 Hz, 2H), 7.64 (s, 1H), 9.08bs, 1H).

Analogous to example 1 39.0 g of compound 102 are obtained by reacting 40.0 g of compound 102a.

¹H-NMR (360 MHz, d₆-acetone) δ=3.80 (s, 6H), 3.83 (s, 6H), 5.98 (s, 2H), 7.21 (d, J=8.5 Hz, 4H), 7.52 (d, J=8.5 Hz, 4H), 7.68 (s, 2H).

### Example 3

### Preparation of the Compound of Formula

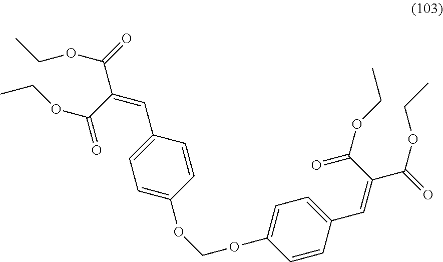

Analogous to example 1 the compound of the formula

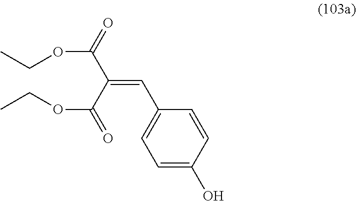

is obtained by Knoevenagel condensation of diethylmalonate (116 g) with 4-hydroxybenzaldehyde (90.0 g) in a yield of 112 g.

¹H-NMR (CDCl₃) δ=1.32 (t, J=7.0 Hz, 6H), 4.29 (q, J=7.5 Hz, 2H), 4.36 (q, J=7.5 Hz, 2H), 6.43 (bs, 1H), 6.78 (d, J=8.5 Hz, 2H), 7.32 (d, J=8.5 Hz, 2H), 7.66 (s, 1H).

Analogous to example 1 compound 103 is obtained in a yield of 39.0 g by reacting 40.0 g of compound 103a.

¹H-NMR (CDCl₃) δ=1.30 (t, J=7.0 Hz, 6H), 1.32 (t, J=7.0 Hz, 6H), 4.29 (q, J=7.5 Hz, 4H), 4.34 (q, J=7.5 Hz, 4H), 5.76 (s, 2H), 7.08 (d, J=8.5 Hz, 4H), 7.43 (d, J=8.5 Hz, 4H), 7.66 (s, 2H).

### Example 4

### Preparation of the Compound of Formula

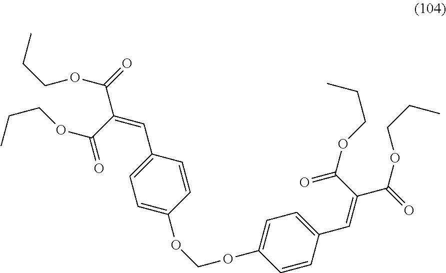

Analogous to example 1 the compound of the formula

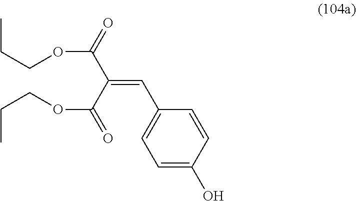

is obtained by Knoevenagel condensation of di-n-propylmalonate (20.0 g) with 4-hydroxybenzaldehyde (10.6 g) in a yield of 13.5 g.

¹H-NMR (CDCl₃) δ=0.93 (t, J=7.0 Hz, 3H), 0.96 (t, J=7.0 Hz, 3H), 1.71 (m_{c}, 4H), 4.19 (q, J=7.5 Hz, 2H), 4.25 (q, J=7.5 Hz, 2H), 6.31 (bs, 1H), 6.79 (d, J=8.5 Hz, 2H), 7.32 (d, J=8.5 Hz, 2H), 7.66 (s, 1H).

Analogous to example 1 compound 104 is obtained in a yield of 12 g by reacting 13.0 g of compound 104a.

¹H-NMR (CDCl₃) δ=0.91 (t, J=7.0 Hz, 6H), 0.97 (t, J=7.0 Hz, 6H), 1.7 (m_{c}, 8H), 4.19 (q, J=7.5 Hz, 4H), 4.23 (q, J=7.5 Hz, 4H), 5.76 (s, 2H), 7.08 (d, J=8.5 Hz, 4H), 7.43 (d, J=8.5 Hz, 4H), 7.66 (s, 2H).

### Example 5

### Preparation of the Compound of Formula

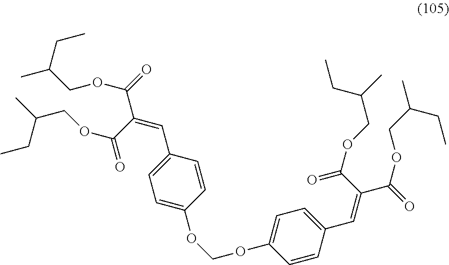

Analogous to example 1 di-(2-methylbutyl)-malonate is obtained by transesterification of di-ethyl malonate with 2-methyl butanol by addition of H₂SO₄ (conc.).

Analogous to example 1 compound of formula

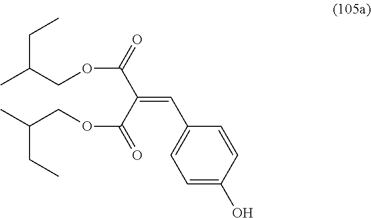

is obtained by Knoevenagel condensation of di-(2-methylbutyl)-malonate (100 g) with 4-hydroxybenzaldehyde (50 g) in a yield of 96 g.

¹H-NMR (CDCl₃) δ=0.86-0.96 (m, 12H), 1.11-1.27 (m, 2H), 1.36-1.51 (m, 2H), 1.74-1.81 (m, 2H), 4.01-4.18 (m, 4H), 6.08 (bs, 1H), 6.79 (d, J=8.5 Hz, 2H), 7.32 (d, J=8.5 Hz, 2H), 7.65 (s, 1H).

Analogous to example 1 compound 105 is obtained in a yield of 12 g by reacting 13.0 g of compound 105a.

¹H-NMR (CDCl₃) δ=0.84-0.95 (m, 24H), 1.09-1.28 (m, 4H), 1.34-1.51 (m, 4H), 1.70-1.82 (m, 4H), 4.01-4.16 (m, 8H), 5.76 (s, 2H), 7.08 (d, J=8.5 Hz, 4H), 7.42 (d, J=8.5 Hz, 4H), 7.66 (s, 2H).

### APPLICATION EXAMPLE

Example B1: O/W sunscreen formulation A

| | Trade Name | INCI-Name | Supplier | % w/w |
|---|---|---|---|---|
| Part A | Amphisol K | Potassium Cetyl Phosphate | Roche | 1.80 |
| | Cutina GMS | Glyceryl Stearate | Cognis | 2.50 |
| | Lanette 18 | Stearyl Alcohol | Cognis | 2.50 |
| | Paraffin | Mineral Oil | Merck | 5.00 |
| | Compound (103) | | | 2.00 |
| | Tegosoft TN | C12-15 Alkyl Benzoate | Degussa | 8.00 |
| | Parsol 1789 | Butyl Methoxydibenzoylmethane | DSM | 2.00 |
| Part B | Water | Aqua | | 65.10 |
| | Rhodicare S | Xanthan Gum | Rhodia | 0.30 |
| | Glycerin | Glycerin | Fluka | 10.00 |
| Part C | Phenonip | Phenoxyethanol (and) Methylparaben | Clariant | 0.80 |
| | | (and) Ethylparaben (and) Butylparaben | | |
| | | (and) Propylparaben (and) Isobutylparaben | | |

Manufacturing Instruction:

Prepare the part A and part B separately and heat to 75° C. Under increasing stirring, incorporate part B to part A and homogenize with Ultra Turrax for 10 sec. at 10000 rpm. Let cool down to room temperature under stirring. At room temperature adjust pH between 5.80 and 6.20.

The sample is applied on sand blasted PMMA plates (delivered by Helioscience, Marseille, France) with a concentration of 1.4 mg/cm², irradiated with an Atlas CPS+ Irradiator and tested in an Optometrics SPF 290 analyzer. The testing procedure is carried out according to DIN 67502. The calculation of the in vitro SPF is done according to M. Wloka et al., Proceedings of the 8^{th} International Conference, The Royal Society, London, Paper12. The in vitro SPF is determined to be 5.2.

The photostability of Butyl Methoxydibenzoylmethane is determined by spreading the emulsion as a 20 μm thick film on a quartz plate. The film is irradiated using a solar simulator for 2 h (5400 kJ/m²). After irradiation, the quartz plate is plunged into 5 ml of tetrahydrofurane. The amount of UV-A filter Butyl Methoxydibenzoylmethane is then determined using high performance liquid chromatography. The residual Butyl Methoxydibenzoylmethane is determined to be 19%.

Example B2: O/W sunscreen formulation B (comparative)

| | Trade Name | INCI-Name | Supplier | % w/w |
|---|---|---|---|---|
| Part A | Amphisol K | Potassium Cetyl Phosphate | Roche | 1.80 |
| | Cutina GMS | Glyceryl Stearate | Cognis | 2.50 |
| | Lanette 18 | Stearyl Alcohol | Cognis | 2.50 |
| | Paraffin | Mineral Oil | Merck | 5.00 |
| | Tegosoft TN | C12-15 Alkyl Benzoate | Degussa | 10.00 |
| | Parsol 1789 | Butyl Methoxydibenzoylmethane | DSM | 2.00 |
| Part B | Water | Aqua | | 65.10 |
| | Rhodicare S | Xanthan Gum | Rhodia | 0.30 |
| | Glycerin | Glycerin | Fluka | 10.00 |
| Part C | Phenonip | Phenoxyethanol (and) Methylparaben | Clariant | 0.80 |
| | | (and) Ethylparaben (and) Butylparaben | | |
| | | (and) Propylparaben (and) Isobutylparaben | | |

The formulation is prepared as described for formulation A.

The photostability of Butyl Methoxydibenzoylmethane in the comparative formulation B is determined as described above for formulation A. The residual Butyl Methoxydibenzoylmethane is determined to be 4%.

This means that the irradiated formulation A comprising Butyl Methoxydibenzoylmethane (BMDBM) and a benzylidene malonate according to the present invention contains significantly higher amounts of BMDBM by a factor 4.75 as compared to the irradiated formulation B which contains BMDBM alone.

1-7. (canceled)

8. A process for the preparation of the compounds of formula (2′), which comprises reacting a malonate (2a) with p-hydroxybenzaldehyde (2b) to a phenol intermediate (2c) in a Knoevenagel condensation (a) and reacting two molecules of this intermediate with a dihaloalkane (2d) to the compound (2′) (step (b)) according to the following reaction scheme: 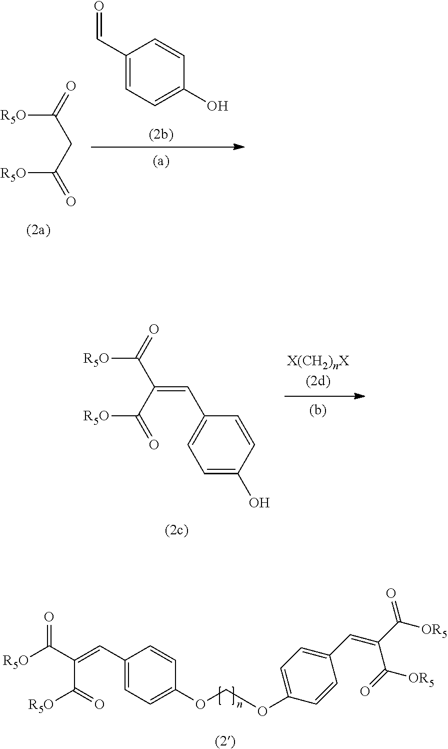
wherein
X is Cl, Br or I;
R₅ is C₁-C₁₈alkyl; unsubstituted or by one or more than one C₁-C₄alkyl substituted C₃-C₁₂-cycloalkyl; unsubstituted or C₁-C₆alkyl-substituted C₆-C₂₀aryl; organosilanyl; or organosiloxanyl; and
n is a number from 1 to 20.

9. A process according to claim 8 further comprising transesterifying a first compound (2′) with an alcohol R₈OH to form a second compound (2′) (step (c)) according to following reaction scheme: 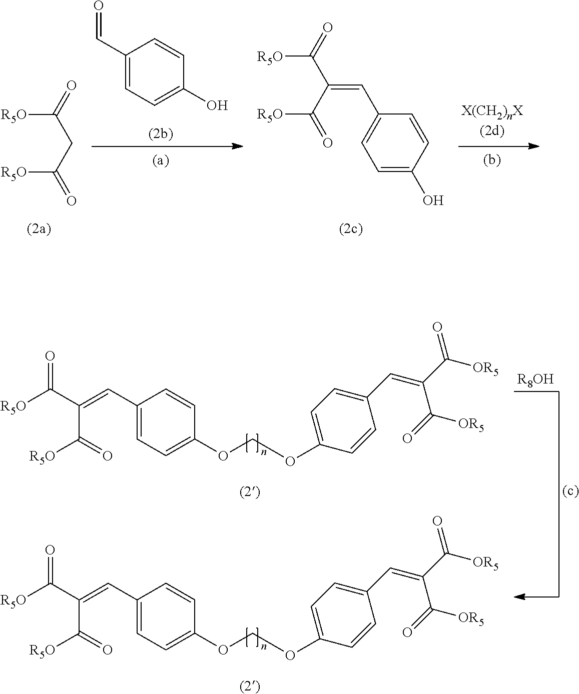
wherein
R₈ independently is defined as is R₅.

10. A process for the preparation of compounds of formula (2′), which comprises reacting two molecules of p-hydroxybenzaldehyde (2b) with a dihaloalkane (2d) to give an intermediate (2e) (step (a)) and subsequent Knoevenagel condensation of this intermediate with a malonate (2a) (step (b)) according to the following reaction scheme: 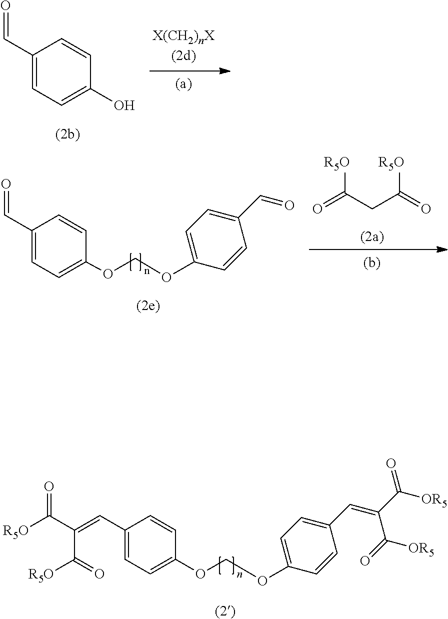
wherein
X is Cl, Br or I;
R₅ is C₁-C₁₈alkyl; unsubstituted or by one or more than one C₁-C₄alkyl substituted C₃-C₁₂-cycloalkyl; unsubstituted or C₁-C₆alkyl-substituted C₆-C₂₀aryl; organosilanyl; or organosiloxanyl; and
n is a number from 1 to 20.

11. A cosmetic preparation comprising one or more compounds of formula (1) together with cosmetically tolerable carriers or adjuvants 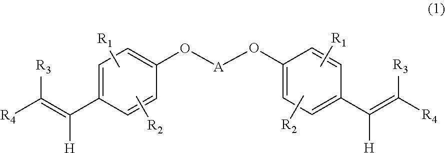
wherein
R₁ and R₂, independently from each other are hydrogen; OH; C₁-C₁₂alkyl; C₁-C₁₂alkoxy; C₁-C₁₂alkylamino; C₁-C₁₂dialkylamino; or water-solubilizing groups selected from carboxylate, sulfonates and ammonium radicals;
R₃ is COOR₅; COR₅; CONR₅R₆; CN; or SO₂R₇;
R₄ is COOR₈; COR₉; CONR₈R₉; CN; or SO₂R₁₀;
R₅, R₆, R₇, R₈, R₉ and R₁₀ independently from each other are C₁-C₁₈alkyl; unsubstituted or by one or more than one C₁-C₄alkyl substituted C₃-C₁₂-cycloalkyl; unsubstituted or C₁-C₆alkyl-substituted C₆-C₂₀aryl; organosilanyl; or organosiloxanyl; and
A is —C₁-C₂₀alkylene-, —CH₂CH(OH)CH₂—, —C₅-C₂₀cycloalkylene, —C₂-C₂₀alkenylene-, —C₅-C₁₀arylene-, or —C₅-C₁₀arylene-(C₁-C₁₀alkylene)- which are unsubstituted or substituted, straight-chain or branched and optionally interrupted by one or more than one heteroatoms selected from —O, —S— and —NH—.

12. A preparation according to claim 11 that comprises further UV protective agents.

13. (canceled)

14. Compounds of formula (2′) 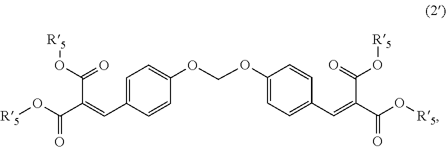
wherein
R′₅ is methyl, propyl, isopropyl, n-butyl, sec-butyl, isobutyl, 2-methylbutyl, 2-ethylhexyl, SiMe₃, CH₂SiMe₃, CH₂CH₂SiMe₃, CH₂CH₂CH₂SiMe₃ or CH₂CH₂CH₂Si(OSiMe₃)₂Me.
